(19) United States
(12) Patent Application Publication
(10) Pub. No.: US 2006/0253176 A1
(43) Pub. Date: Nov. 9, 2006

(54) Dermatological treatment device with deflector optic

(75) Inventors: Joseph P. Caruso, Reading, MA (US); Gregory B. Altshuler, Lincoln, MA (US); Liam O'Shea, Medford, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: PALOMAR MEDICAL TECHNOLOGIES, INC., Burlington, MA (US)

(21) Appl. No.: 11/357,749

(22) Filed: Feb. 17, 2006

Related U.S. Application Data
(60) Provisional application No. 60/654,123, filed on Feb. 18, 2005.

(51) Int. Cl. A61N 5/06

(52) U.S. Cl. 607/88

(57) ABSTRACT

This invention relates generally to methods and apparatus for utilizing energy, e.g., optical radiation, to treat various dermatological and cosmetic conditions. A handheld dermatological device that facilitates viewing and measuring parameters of a treatment area before, during, and after application of a treatment modality, and methods of use therefor, are disclosed. The device can include a deflective device that can steer radiation to control a target position of the radiation. The device can also include a control device to allow a user to control the radiation through manipulation of the deflective device.

### RELATED APPLICATIONS

This application claims benefit of priority to U.S. Provisional Application No. 60/654,123, filed Feb. 18, 2005, the entire disclosure of which is incorporated herein by reference.

### TECHNICAL FIELD

This invention relates generally to methods and apparatus for utilizing energy, e.g. optical radiation, to treat various dermatological and cosmetic conditions.

### BACKGROUND OF THE INVENTION

Energy such as electromagnetic, mechanical, thermal, acoustic, and particle beam radiation has been utilized for many years in medical and non-medical facilities to treat various medical and cosmetic conditions. Such treatments include, but are not limited to, hair growth management, including limiting or eliminating hair growth in undesired areas and stimulating hair growth in desired areas, treatments for PFB (razor bumps), skin rejuvenation, anti-aging treatments including improving skin texture, elasticity, wrinkles and skin lifting and tightening, pore size reduction, reduction of non-uniform skin pigmentation, improving vascular and lymphatic systems, treatment of vascular lesions such as spider veins, leg veins, varicose veins, port wine stain, rosacea, telangiectasia, removal of pigmented lesions, repigmentation, improved skin moistening, treatment of acne including non-inflammatory, inflammatory and cysts, treatment of psoriasis, reduction of body odor, reduction of oiliness, reduction of sweat, reduction/removal of scars, prophylactic and prevention of skin diseases, including skin cancer, improvement of subcutaneous regions, including fat reduction and cellulite reduction, as well as numerous treatments for other conditions.

The treatments can be performed, for example, by employing optical energy (including ultraviolet, visible, and infrared), microwave energy, radiofrequency, low frequency or DC current energy, acoustic energy, mechanical energy and kinetic energy of particles (for example, sapphire particles), skin cooling or heating. The flow of energy can be delivered to the treatment region via a handpiece, which can include a housing, energy distribution system (comprising, for example, a radiation source, optics and a scanner), and an optional skin cooling element. In rare cases, the handpiece can also include a diagnostic sensor (i.e., skin temperature radiometer). The diagnostic sensor in such systems is used to protect the skin from unwanted damage (i.e., due to overheating or over cooling).

While various handheld devices have been disclosed for applying dermatological treatments, currently, present systems lack efficient mechanisms for positioning the treatment head of the handpiece over a selected target treatment area and/or viewing the target area while a treatment modality is being applied. Further, such conventional handheld devices lack systems for preferential imaging of subsurface skin tissue.

Accordingly, there exists a need for handheld dermatological devices that provide mechanisms for positioning the device's treatment head over a target area and/or viewing the target area even as the treatment is being applied.

There is also a need for such handheld dermatological devices that can provide better targeting and evaluation of a treatment target and surrounding tissue before, during, and after treatment to improve efficacy and safety of the treatment and provide an opportunity for self treatment with a cosmetic device suitable for home use.

### SUMMARY OF THE INVENTION

Methods and devices for treating dermatological or cosmetic conditions that include imaging a target skin region, e.g., skin tissue, are disclosed that allow preferential illumination of the skin region, obtaining its image and displaying it to a user for better alignment of flow of a treatment energy relative to a target region and performing diagnostic of the target before, during and after treatment. A user of such devices can be, for example, a physician, an aesthetician, or a person who can utilize the device for self-treatment of a cosmetic condition. In some embodiments, the device provides diagnostic functionality. In some embodiments, the devices include handheld devices that can, in addition to imaging capability, provide treatment energy to a subject's skin.

According to one aspect, the invention includes a handheld dermatological device having a deflective device, such as a deflective optic, that can steer a laser beam to control a target position of the laser beam. The deflector can comprise, for example, an optical device or an electro-optic positioning device. In some embodiments, the deflector comprises a mechanical motor and an optic device, wherein the mechanical motor can adjust an angular position of the optic device to steer radiation. The deflector can be disposed in an optical path between the radiation source and the portion of the housing. The handheld device can include a control device to allow the user to control the laser beam through manipulation of the deflective optic. In this embodiment, the user need not readjust the position of the handheld device on the subject's skin to treat a different area of the skin. Instead, by controlling a target position of the laser beam, the user can treat any area within a treatment window (target treatment area) of the device without adjusting the position of the device. The control for the laser can, in one embodiment, be in the handheld device. In other embodiments, the control can be remotely located from the handheld device. The control device can be, for example, a joystick, a roller, a touch pad, and a keypad.

As used herein, the term “treatment energy” can refer to therapeutic energy to treat a diseased condition or energy suitable for treating cosmetic conditions.

In one aspect, a handheld dermatological device is disclosed that allows application of treatment energy to a target skin region as well as visualizing the skin treatment region prior to, during, and/or after application of the treatment energy. Such a device can include, for example, an image capture device and a display device that is mounted to a housing of the handheld device and is coupled (electrically, optically or otherwise (e.g. wirelessly)) to the image capture device to present acquired images to a user, e.g., a medical professional or a consumer. The image capture device can be connected to the display through a microprocessor, which can be integrated with the display or the image capture device itself. The handheld device can further include a head that can be precisely aligned by a user relative to a patient's skin by utilizing one or more images presented in the display, and through which treatment energy can be applied to a target skin region.

A dermatological device, as used herein, can refer to a therapeutic device or a cosmetic device, including a home cosmetic device.

In one aspect, a handheld dermatological device is disclosed that includes a housing capable of being manually manipulated to position a head portion thereof in proximity to a person's skin, and is adapted for delivery of treatment energy to a target skin region. The handheld device can further include an illumination source coupled to the housing for generating radiation to illuminate the target skin region, and a detector disposed in the housing and adapted to primarily detect tissue scattered radiation emanating from the target skin region. The illumination source can be an illumination ring.

As used herein, the term “primarily detect tissue scattered radiation emanating from the target skin region” is intended to mean detecting radiation from the illumination source that is primarily scattered by tissue below and around the target skin region depth and thus reaches the detector from beneath the skin surface thereby emanating or coming from the target skin region. This may also be referred to as “translucent radiation” where the radiation is coming from below the target skin region to make the target skin region more visible. The term “primarily” in this context is used to distinguish between such tissue scattered radiation and light that reaches the detector by reflection of illuminating or ambient light from the surface of the skin and skin above the target skin region. Thus, “primarily” typically means greater than 50%, greater than 60%, greater than 70%, greater than 80%, greater than 90%, or greater than 95%, of the detected radiation corresponds to scattered radiation emanating from below and around the target skin region depth as opposed to light reflected by the skin surface and scattered from the skin above depth of target skin region.

The detector can be positioned relative to an illumination source so as to primarily detect the scattered radiation. The detector can optionally include an image capture device for generating an image of the target skin region. Further, a display can be mounted to the housing for displaying the image. In some embodiments, the detector is an image capture device for generating an image of the illuminated tissue in the target treatment area. The image capture device can be, for example, a CCD camera or an electro capacitor image capture device. The image capture device can include a display device to display the image. The display device can be mounted on the housing, moveably coupled to the housing, or wirelessly coupled to electronics within the housing.

In some embodiments, the apparatus further includes an image processor in communication with the image capture device and the display device. For example, microprocessor or a memory element can be in communication with the image capture device. The memory element can store software for controlling the deflector to selectively irradiate treatment locations within the target treatment area.

The illumination source can be adapted to deliver radiation to a first skin surface segment so as to illuminate the target region such that at least a portion of the scattered radiation reaches the detector via a second skin surface segment. A shield mounted to the head portion can shield the second skin surface segment from direct (via skin surface) application of radiation from the illumination source. In some embodiments, the device can further include additional illumination sources. In certain embodiments, the illumination sources can be selected to generate radiation with different wavelengths. A control unit can be further included for selectively activating at least one, or more of the illumination sources. For example, the control unit can activate the illumination sources according to a preset temporal pattern.

The illumination source can comprise, for example, a laser or a lamp. In some embodiments, the apparatus further comprising a lens optically coupled to the laser. The lens can be a zoom lens. In some embodiments, the apparatus can include one or more sensors mounted on a head portion of the housing. The sensors are capable of generating a dielectric image of the target treatment area.

In some embodiments, the housing can include an aperture through which the scattered radiation can reach the detector. The illumination source is preferably offset relative to the aperture such that illuminating radiation reaches the target region along different paths than those along which scattered light from the target region is collected by the detector. In some embodiments, the illumination source can be positioned at an angle relative to an optical axis of the device.

In other aspects, a treatment source can be disposed in the housing for generating the treatment energy. By way of example, the treatment source can generate electromagnetic radiation (EMR) having one or more wavelengths in a range of about 290 nm to about 3000 nm, in a range of about 500 to about 3000 nm, in a range of about 600 nm to about 1900 nm, or in a range of about 800 nm to about 1100. The treatment source can generate pulsed radiation having a fluence in a range of about 1 to about 200 J/cm² with pulse widths in a range of about 1 ns to about 10 seconds. For example, the treatment source can be a neodymium (Nd) laser, such as a Nd:YAG laser.

In another aspect, the housing can be adapted for receiving the treatment energy from an external treatment source, such as a radiation source. For example, the device can further include one or more optical fibers for directing radiation from an external treatment source to the target skin region. Optical fiber can be utilized for delivery of illumination light from illuminating sources to the skin.

In further aspects, the device can further include a first polarizer coupled to the illumination source and a second polarizer coupled to the detector. In some embodiments, the polarizers have substantially orthogonal or parallel polarization axes.

In another aspect, a method of treating a target skin region is disclosed comprising illuminating the target skin region, detecting primarily tissue scattered radiation emanating from the target region in response to the illumination, and directing treatment energy to at least a portion of the target skin region. A first portion (segment) of skin surface can be illuminated with illuminating radiation propagating along a first direction such that at least a portion of the radiation penetrates the skin tissue below a second portion (segment) of skin surface. The second portion (segment) of the skin surface can be shielded from direct application of the radiation. The scattered radiation can be detected along a second direction offset relative to the first direction. The radiation emanating from the second segment of skin surface can be detected to obtain an image of the target skin region. The image can be used to align a treatment energy beam with a portion of skin tissue in the target skin region so as to apply treatment energy to that portion. By way of example, the illumination radiation can be selected to have one or more wavelengths in a range of about 290 nm to about 3000 nm. One or more images of the target skin region can be monitored before, during, or after application of the treatment energy.

In another aspect, a handheld dermatological device is disclosed comprising a housing capable of being manually manipulated to position a head portion thereof in proximity to a person's skin surface, an illuminating source mounted to the housing for illuminating a target skin region, a neodymium (Nd) laser, e.g., a Nd:YAG laser, disposed in the housing for generating radiation, an optical system coupled to the laser for directing radiation from the laser to the target skin region, an image capture device mounted in the housing for generating an image of the illuminated target skin region, and a display coupled to the housing and in communication with the image capture device to display the image. The device can further include a shield mounted to the head portion for shielding a portion of a skin surface through which the image capture device obtains an image of the target skin region from direct illumination by the illumination source. A zoom lens system coupled to the laser can adjust a dimension, e.g., a diameter, of a radiation beam generated by the laser. The device can further include a microprocessor in communication with the image capture device for processing one or more images obtained by the image capture device. The image capture device can be a CCD camera, a video camera or any other suitable analog or digital imaging system. The device can also include imaging optics optically coupled to the image capture device for directing at least a portion of radiation emanating from the target skin region to the image capture device. In some embodiments, the device further includes additional illumination sources. In some cases, the illumination sources can generate radiation with different wavelengths. A control unit can be further included for selectively activating the illumination sources according to a desired temporal pattern so as to illuminate the target region with radiation having different wavelengths and/or from different angles.

As used herein, the term “treating skin” is intended to encompass both medical and cosmetic treatments, such as hair removal, hair growth management, removal of vascular lesions (e.g., telangiectasia, psoriasis, rosacea, spider vein, leg vein), pigmented lesions, treatment of nail disorders, fat reduction, acne treatment, skin rejuvenation, wrinkle reduction and tattoo removal and the like. The image can be displayed before, during, or after application of the treatment radiation. The image can be used to align a treatment radiation beam with the selected vasculature.

In another aspect, a handheld dermatological device is disclosed comprising a housing through which treatment energy can be applied to a skin target region, an illumination source coupled to the housing for illuminating the target region, an image capture device mounted in the housing for acquiring one or more images of the target region, goggles suitable for wearing by an operator of the device. The goggles can incorporate one or more display devices in communication with the image capture device for displaying the images to the operator. The device can include a treatment source disposed in the housing. Alternatively, the housing can be adapted to receive the treatment energy from an external source, e.g., via an optical fiber or other energy delivery systems.

In another aspect, a method is disclosed for treating a target region of skin tissue comprising illuminating a first skin surface with radiation such that at least a portion of the radiation penetrates the skin tissue below a second skin surface, shielding the second skin surface from direct application of the radiation, detecting radiation emanating from the second skin surface to obtain an image of the target skin region, and directing treatment energy to the target skin region through the second skin surface.

In another aspect, a dermatological device is disclosed comprising a housing through which treatment energy can be applied to a target skin region, a radiation guiding element, such as an optical coupling element, coupled to the housing and adapted to contact a skin surface region, at least one illumination source optically coupled to the coupling element for coupling radiation into the guiding element so as to generate illumination electromagnetic radiation (wave) refractively coupled to at least a portion of skin in contact with the guiding element, and an image capture device capable of detecting radiation scattered from the target region in response to the refractively coupled illumination radiation. The image capture device can form an image of the target region. The radiation guiding element can be formed of any suitable transparent material as discussed in more detail below. For example, the radiation guiding element can be formed of sapphire or quartz and can have an index of refraction in a range of about 1.3 to about 1.9. In another aspect, images exhibiting interruptions of total internal reflection of illumination light at the contact surface of the guiding element and skin surface can be used for visualization of targets on the skin surface. The device can further comprise a polarizer coupled to the image capture device so as to prevent radiation having a selected polarization from reaching the image capture device. A filter coupled to the image capture device can be included in the device so as to prevent radiation having one or more selected wavelengths from reaching the image capture device.

In another aspect, a method of treating a person's skin is disclosed comprising placing an optical coupling element on a portion of the skin surface, coupling illuminating radiation into the guidance element to generate refractively coupled waves penetrating a subsurface region below the portion of the skin surface, detecting at least a portion of radiation scattered by the subsurface region in response to the refractively coupled waves to form an image of the subsurface region, and directing treatment energy to at least a portion of the subsurface region.

In a related aspect, radiation can be coupled to the guidance element so as to generate evanescent waves at the interface of the guidance element with the skin. Such waves can be utilized for imaging and diagnosis of dermatological structures and conditions, as discussed in more detail below.

In another aspect, a handheld dermatological device is disclosed comprising a housing through which treatment energy can be applied to a person's skin, two illumination sources capable of generating radiation having at least two different wavelengths, the sources being mounted to a head portion of the housing for illuminating a target skin region, a control unit for selectively activating the sources, and an image capture device disposed in the housing for detecting at least a portion of radiation scattered by the target skin region in response to illumination by at least one of the sources. The control can be adapted for activating the sources in different temporal intervals and/or for triggering the image capture device to form an image of the target region upon activation of at least one of the sources. The device can include a shield positioned in proximity of at least one of the illumination sources for shielding a selected skin surface segment from direct illumination by that source. The image capture device can be adapted to collect radiation via the shielded skin surface segment radiation scattered by the target skin region. The device can further include a treatment source disposed in the housing for applying treatment energy to the target skin region through the shielded skin surface segment.

In another aspect, the invention provides a device for imaging a subsurface target region of skin tissue that includes an illumination source for illuminating a skin surface with illuminating radiation such that at least a portion of the radiation penetrates the skin tissue below the surface and is at least partially scattered by the skin tissue. The device can further include a detector that is capable of detecting radiation scattered by the subsurface target region, and a shield for shielding the detector from illuminating radiation that is directly reflected by the skin surface. The detector can comprise an image capture device that can generate an image of the subsurface target region. Any suitable image capture device can be employed. For example, the image capture device can be a CCD/CMOS camera or a video camera.

The device can include a handheld housing through which treatment energy can be directed to the skin. The treatment energy can be provided by a source mounted to the housing, or alternatively, it can be provided by an external source and guided through a path within the housing to the skin. In some embodiments, the treatment source is a radiation source, such as a laser or a broad band source (e.g., a lamp, a LED).

In a related aspect, in the above imaging or the handheld device, the shield can comprise a polarizer coupled to the detector to prevent radiation having a selected polarization direction from reaching the detector. In some embodiments, another polarizer having a polarization axis orthogonal or parallel to the shield polarizer, can be coupled to the illumination source. Alternatively, the shield can be formed from a material that is substantially opaque to the radiation generated by the illumination source, and can be placed in proximity of the illumination source to prevent direct illumination of a portion of the skin surface of the target region.

In further aspects, the invention provides a method for imaging a subsurface target region of skin tissue that includes illuminating a skin surface with illumination radiation such that a significant portion of the radiation penetrates the skin tissue below the surface and is at least partially scattered by that tissue while minimizing scattering signal from skin tissue located deeper than the target tissue. A detector is positioned so as to detect at least a portion of radiation scattered by the subsurface target region. The detector is shielded from illumination radiation that is directly reflected by the skin surface (and scattered from tissue above the target region depth) while enhancing detection of radiation that is primarily scattered by tissue below and around target skin region depth, and an image of the subsurface target region is obtained based on the detected scattered radiation. The illumination radiation can have one or more wavelengths in a range of about 350 nm to about 2000 nm. The illumination sources can be, for example, light emitting diodes (LED), diode lasers, lamps, or other suitable sources of electromagnetic energy. In some cases, treatment energy, e.g., radiation having one or more wavelengths in a range of about 290 nm to about 1,000,000 nm, can be applied to the subsurface target region in conjunction with monitoring one or more images of this region prior to, during, and/or after application of the treatment energy.

In a related aspect, the invention provides a handheld dermatological device that includes a housing capable of being manually manipulated to direct treatment energy to a skin target region, an image capture device coupled to the housing to generate an image of at least a portion of the target region, and a display device mounted to the housing and electrically coupled to the image capture device to display images captured by the image capture device.

The term “mounted,” as used herein, is intended to encompass mechanical coupling to the housing such that the housing and the display can be simultaneously, or separately, manually manipulated by the user to direct treatment radiation to a target area and/or view the target.

The housing can further comprise a head capable of transmitting the treatment energy. The user can precisely position the head over a desired portion of the treatment region by using the display as a guide. The user can therefore more effectively diagnose and/or view the treatment region before, during and/or after treatment. Thus, more effective and safer treatment will be possible than are currently available as the user can directly monitor the results of the treatment in real-time.

In some embodiments, the handheld device can include an optical system, such as an objective, optical filter, spectral filter, spatial filter, polarizer, phase element, mask and illumination system for facilitating acquisition of images and/or enhancing their presentation. For example, such an optical system can be disposed between the image capture device and the patient's skin to prevent radiation having selected wavelengths and/or polarizations from reaching the image capture device.

Further, an image of the treatment region can be processed by a microprocessor, for example, to enhance its resolution (or contrast), color and brightness. For example, the microprocessor can be positioned between an image capture device and a display. In some embodiments, the microprocessor can be coupled to the image capture device such that the user can be alerted when a treatment has reached a desired preset limit. The microprocessor can provide image processing for magnification, improved contrast of the image, and/or synchronization of the image capture with skin illumination, as discussed in more detail below. For example, the image capture device can send multiple images of the treatment region during treatment to the microprocessor. The microprocessor can compare changes in selected parameters of the treatment region to threshold values previously stored, for example, in a database stored in a memory element. Various parameters, such as color or a change of fluorescence emission, can be used to monitor the applied treatment. Skin conditions, such as, pigmented lesions, spider veins, port wine stains, psoriasis, can change color during and after treatment. The treatment radiation can also coagulate and/or destroy vessels resulting in a color change in images of such vessels. Additionally, treatment of acne can be monitored through a measurement of fluorescence. Among microbial population of pilosebaceous unit, most prominent is Propionibacterium Acnes (P. Acnes). These bacteria are causative in forming inflammatory acne. P. Acnes can exhibit fluorescence. Upon treatment, the fluorescence will decrease.

Images and/or other data of the treatment region can be stored in a memory element, such as a chip or a memory card, which can be attached to the microprocessor, or sent to a computer via a wireless or hard-wired connection. These images and/or data can be used, for example, to compile a patient or treatment history file.

In some embodiments, the display device can be fixedly mounted onto the housing. In other embodiments, the display device can be moveably mounted onto the housing. In yet other embodiments, the display device can be hingedly attached to the housing. For example, the display device can be attached to a railing or flexible wire such that the display device can be extended by the user for ease of viewing and can be folded for ease of storage. Such an adjustable display device can be utilized, for example, by a patient for self-treatment In other aspects, the displays can be built into goggles to be worn by a user or a patient. The display device can be permanently attached to the housing of the handheld device, or it can be mounted to the housing in a removable and replaceable manner. In some embodiments, a large display can be used for providing better image resolution, and facilitating simultaneous observation of an image by an operator and a patient. In other embodiments, the display can be physically separate from the housing and be connected to the housing using a wireless connection.

The image capture device can detect a change in at least one of optical signals, infrared, electro capacitance, or acoustic signals. An electro capacitor image capture device can be desirable for skin surface and epidermis imaging. The image capture device can be either an analog or a digital device. In some embodiments, the image capture device is a camera. In certain embodiments, the image capture device is a CCD/CMOS camera or a video camera.

A handheld dermatological device according to the teachings of the invention can be utilized to deliver different types of treatment energy to a patient. Some exemplary optical radiation wavelengths and examples of conditions that can be treated by these wavelengths are provided in Table 1 below.

In another embodiment, the invention discloses a handheld dermatological device for performing a treatment on tissue including a means for housing components, the housing means having a portion that defines a target treatment area on the tissue when located in proximity to the tissue, a means for generating radiation, and a means for selectively steering radiation from the means for generating radiation to a treatment location within the target treatment area. The device can further include a means for controlling the means for selectively steering.

In another aspect, the invention discloses a method of operating a handheld dermatological device comprising capturing an image of a condition on tissue through an image capture device of the handheld dermatological device, wherein the image of the condition is of at least a portion of the condition in a target treatment area defined by a head portion of the handheld dermatological device when located in proximity to the tissue; and steering radiation to selectively treat the condition within the target treatment area. Radiation can be steered through adjustment of a deflector. The radiation can be from a radiation source of the handheld dermatological device. The method can further include controlling the steering of the radiation through a user control device and/or displaying the image to an operator of the handheld dermatological device. The displaying the image can include displaying a graphic to represent where the radiation will strike the tissue. The deflector can comprise, for example, an electro-optic positioning device, or a mechanical motor and an optic device, wherein the mechanical motor can adjust an angular position of the optic device to steer radiation. The deflector can be disposed in an optical path between the radiation source and the portion of the housing.

In some embodiments, the method further comprising displaying an image of the condition to an operator of the handheld dermatological device, illuminating the target treatment area to aid in capturing the image, and/or calculating positional information of the condition within the target treatment area. The method can also include calculating adjustment information for the deflector based on the positional information, selectively controlling firing of a radiation source to treat the condition, and/or irradiating multiple treatment locations within the target treatment area upon selective steering of the deflector.

In another aspect, a method of automatically operating a handheld dermatological device is disclosed, comprising capturing an image of a condition on tissue through an image capture device of the handheld dermatological device, wherein the image of the condition is of at least a portion of the condition in a target treatment area defined by a head portion of the handheld dermatological device when located in proximity to the tissue; calculating positional information of the condition within the target treatment area; calculating adjustment information for a deflector of the handheld dermatological device based on the positional information; and steering radiation in accordance with the adjustment information to treat the condition within the target treatment area. The method can further include displaying an image of the condition on a display of the handheld dermatological device. The firing of a radiation source to treat the condition can also be selectively controlled.

In yet another aspect, a method of treating a target skin region is disclosed, comprising applying radiation to the target skin region; generating an image of the target skin region; displaying the image of the target skin region; and steering the radiation to selectively irradiate the target skin region. The method can further include controlling the steering of the radiation through a user control device.

TABLE 1
Exemplary parameters for the treatment of dermatological conditions
with light.

| Treatment condition or application | Wavelength, nm |
|---|---|
| Anti-aging | 400-11000 |
| Superficial vascular | 290-600 |
| | 1300-2700 |
| Deep vascular | 500-1300 |
| Pigmented lesion, de pigmentation | 290-1300 |
| Skin texture, stretch mark, scar, porous | 290-2700 |
| Deep wrinkle, elasticity | 500-1350 |
| Skin lifting | 600-1350 |
| Acne | 290-700, 900-1850 |
| Psoriasis | 290-600 |
| Hair growth control, | 400-1350 |
| PFB | 300-400, 450-1200 |
| Cellulite | 600-1350 |
| Skin cleaning | 290-700 |
| Odor | 290-1350 |
| Oiliness | 290-700, 900-1850 |
| Lotion delivery into the skin | 1200-20000 |
| Color lotion delivery into the skin | Spectrum of absorption of color |
| | center and 1200-20000 |
| Lotion with PDT effect on skin | Spectrum of absorption of photo |
| condition including anti cancer effect | sensitizer |
| ALA lotion with PDT effect on skin | 290-700 |
| condition including anti cancer effect | |
| Pain relief | 500-1350 |
| Muscular, joint treatment | 600-1350 |
| Blood, lymph, immune system | 290-1350 |
| Direct singlet oxygen generation | 1260-1280 |

In embodiments in which the treatment energy is applied as pulses, the pulsewidths can be in a range of about 1 nanosecond to about 10 seconds and the pulses can have a fluence in a range of about 1 to about 200 J/cm².

In other aspects, the invention provides a dermatological imaging device that includes a radiation guiding element (such as an optical coupling element) that is adapted to contact a skin surface region to provide refractive coupling of light into the skin (refractive illumination). The device can further include at least one illumination source that is optically coupled to the coupling element for coupling radiation into the coupling element so as to generate electromagnetic waves penetrating into a controlled depth of subsurface skin region. The device also includes an image capture device that is capable of detecting radiation scattered from the subsurface skin region in response to the refractive wave illumination. The image capture device can form an image of the subsurface skin region by employing the detected radiation. Further, in some embodiments, a filter and/or a polarizer can be coupled to the image capture device to prevent radiation having a selected polarization, or one or more selected wavelengths, from reaching the image capture device. The refractive coupling of radiation into the skin can be utilized for precise control of treatment and/or imaging of skin surface conditions and/or features, such as, stratum corneum structure, pores, sebaceous follicle openings, hair follicle openings, skin texture, wrinkles, psoriasis. By controlling the refractive index of the guiding element and the incident angle of radiation coupled into the coupling element at the contact surface of the coupling element and the skin, the imaging contrast of a visualized target can be enhanced, as discussed in more detail below.

In further aspects, the invention provides a handheld dermatological device that includes a housing through which treatment energy can be applied to a patient's skin, and further includes one or more sensors mounted to a head portion of the housing, which are capable of generating a dielectric image of a target skin region. Such a dielectric image can provide a distribution of dielectric sensitivity of the skin surface of a target skin region, which can be measured, e.g., by an electro capacitor image capture device. The device can further include a display for displaying the dielectric image. In some embodiments, one or more transducer elements can be coupled to the housing for applying an electric current or acoustic energy to the patient's skin.

In another aspect, the invention provides a handheld dermatological device having a housing through which treatment energy can be applied to a patient's skin, and two or more illumination sources that generate radiation having wavelengths in different wavelength bands. The sources are mounted to a head portion of the housing for illuminating a target skin region. The handheld device can further include a control for selectively activating the sources and a image capture device disposed in the housing for detecting at least a portion of radiation scattered by the target skin region in response to illumination by one or both of the sources.

Further understanding of the invention can be obtained by reference to the following detailed description in conjunction with the associated drawings, described briefly below.

### BRIEF DESCRIPTION OF THE DRAWINGS

FIG. 1A schematically depicts a handheld device according to one embodiment of the invention,

FIG. 1B schematically depicts a handheld device according to one embodiment of the invention having a display movably mounted to the device's housing,

FIG. 1C schematically illustrates a handheld device according to one embodiment of the invention having a display electrically or optically coupled to the device's housing via one or more flexible wires or optical cables,

FIG. 1D schematically illustrates a handheld device according to one embodiment of the invention having a display hingedly attached to the device's housing,

FIG. 1E schematically depicts a handheld device according to one embodiment of the invention having a display incorporated in goggles suitable for wearing by a user,

FIG. 2A schematically depicts a handheld device according to one embodiment of the invention having a housing through which therapeutic energy can be applied to the skin and an image capture device for generating an image of a target skin region,

FIG. 2B schematically depicts a handheld device according to another embodiment of the invention having an illumination source for illuminating a target skin region and an image capture device for acquiring an image of the target region,

FIG. 2C schematically illustrates a device according to another embodiment of the invention having an illumination source and an image capture device for preferentially illuminating and obtaining an image of a target skin region,

FIG. 2D schematically illustrates a device according to an embodiment of the invention having a cooling or heating element for applying heat to or extracting heat from the skin and an image capture device for generating an image of a target skin region,

FIG. 2E schematically illustrates a device according to another embodiment of the invention having a plurality of radiation sources adapted for preferentially illuminating a subsurface skin region and an image capture device for generating an image of that region,

FIG. 3A schematically illustrates a device according to one embodiment of the invention in which polarized radiation is employed for preferential illumination of a target skin region,

FIG. 3B schematically illustrates a device according to one embodiment of the invention having a plurality of illumination sources for illuminating a subsurface skin region and a shield disposed in proximity of the sources for shielding a selected skin surface from direct illumination by the sources,

FIG. 4A is a schematic cross-sectional view of a handheld dermatological device according to one embodiment of the invention,

FIG. 4B is a schematic cross-sectional view of a head portion of the device of FIG. 4A,

FIG. 4C schematically depicts illumination sources and a shield mounted to the head portion of the handheld device of FIGS. 4A and 4B,

FIG. 5 schematically depicts an image of a skin portion obtained by an image capture device incorporated in a handheld device according to one embodiment of the invention,

FIG. 6 schematically depicts illumination sources and a shield mounted to a head portion of a device according to one embodiment of the invention in which the sources provide radiation in different spectral bands,

FIG. 7 is a diagram depicting a control system for selective activation of illumination sources and/or an image capture device incorporated in a handheld device according to one embodiment of the invention,

FIG. 8 schematically depicts a handheld device according to one embodiment of the invention having a CCD camera and an image processor for processing images acquired by the camera,

FIG. 9 schematically depicts a handheld device having a communications module for transmitting data obtained by an image capture device incorporated in the handheld device to an external computing system, via wired or wireless communication,

FIG. 10 schematically depicts an image of a target region presented in a display of a handheld device according to one embodiment of the invention in which a graphical object in employed to show a cross-section of a treatment beam,

FIG. 11A schematically illustrates a handheld device according to one embodiment of the invention in which a command menu can be presented to a user,

FIG. 11B schematically illustrates a handheld device according to another embodiment having a microprocessor in communication with an image capture device to process images acquired by the device so as to identify occurrence of a selected condition, such as completion of a treatment protocol,

FIG. 12 schematically illustrates tracking the position of a marker identifying the location of a selected site in two images, which are shifted relative to one another,

FIG. 13A schematically illustrates a handheld device according to another embodiment of the invention having an optical coupling element and an illumination source coupled to the coupling element so as to generate refractively coupled illumination waves for illuminating a subsurface skin region and an image capture device for generating an image of that region,

FIG. 13B schematically illustrates the device of FIG. 13A in which total internal reflection at an contact surface of the optical coupling element and the skin surface is employed for visualizing the skin surface,

FIG. 14A schematically illustrates a device according to one embodiment of the invention having an array of sensors for generating a dielectric image of a skin portion and a display for displaying that image,

FIG. 14B schematically illustrates a device according to one embodiment of the invention that includes, in addition to sensors for generating a dielectric image of a skin portion and a display for displaying that image, one or more transducer elements for applying energy to the skin,

FIG. 15 is a schematic cross-sectional view of a handpiece dermatological device according to one embodiment of the invention having a housing to which a waveguide is coupled to transmit energy from a remote source to a skin portion,

FIG. 16A is a schematic cross-sectional view of a handpiece device according to another embodiment of the invention having a therapeutic radiation source and an illumination radiation source,

FIG. 16B is a schematic cross-sectional view of a handheld device according to one embodiment of the invention having an image capture device for generating an image of a target skin region and a memory unit for storing the images,

FIG. 16C schematically depicts a handheld device according to one embodiment of the invention having a housing in which various components of the device are disposed,

FIG. 16D schematically depicts a handheld device according to another embodiment of the invention,

FIG. 17 schematically depicts a handheld device according to another embodiment of the invention having a source for generating therapeutic energy and a beam forming system for focusing the therapeutic energy onto a selected target skin region,

FIG. 18 schematically illustrates a handheld device according to another embodiment of the invention having a lamp source for generating treatment radiation,

FIG. 19 is a schematic cross-sectional view of a handheld dermatological device according to another embodiment of the invention,

FIG. 20 is a schematic cross-sectional view of the left-hand side of the handheld dermatological device of FIG. 19,

FIG. 21 is a schematic view of the left-hand side of the handheld dermatological device of FIG. 19 in use,

FIG. 22A is a first view of a target treatment area of a subject's skin during use of the dermatological device of FIG. 19,

FIG. 22B is a second view of the target treatment area of a subject's skin during use of the dermatological device of FIG. 19,

FIG. 22C is a third view of the target treatment area of a subject's skin during use of the dermatological device of FIG. 19, and

FIG. 23 is a flow chart of treatment of tissue during use of the dermatological device of FIG. 19 according to one embodiment of the invention.

### DETAILED DESCRIPTION OF THE INVENTION

The present invention relates generally to dermatological devices, and more particularly to handheld dermatological devices for applying a variety of treatment modalities to a patient's skin or any other tissue while allowing a user to view the treatment area and target before, during, and after application of the treatment. In some embodiments, the handheld device can include one or more radiation sources for illuminating a target region of the patient's skin so as to facilitate imaging that region by an image capture device, and can further include a display in which an image of the target region can be presented.

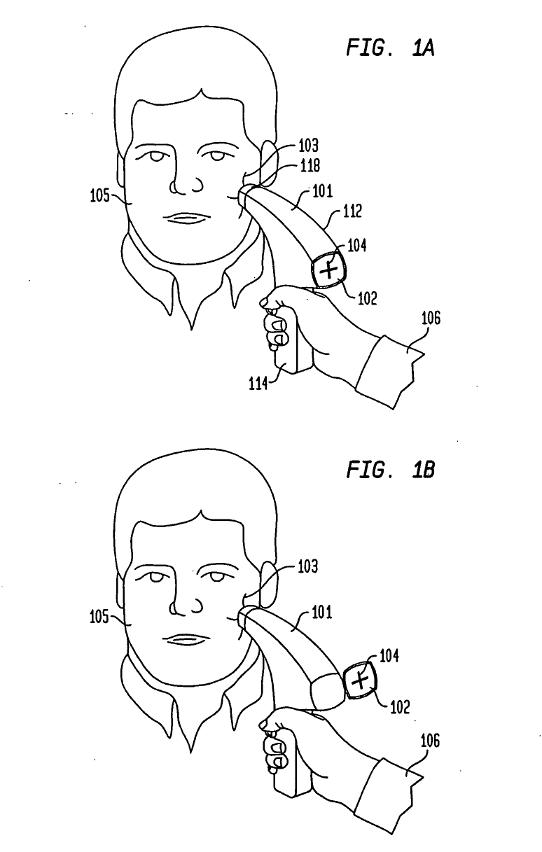

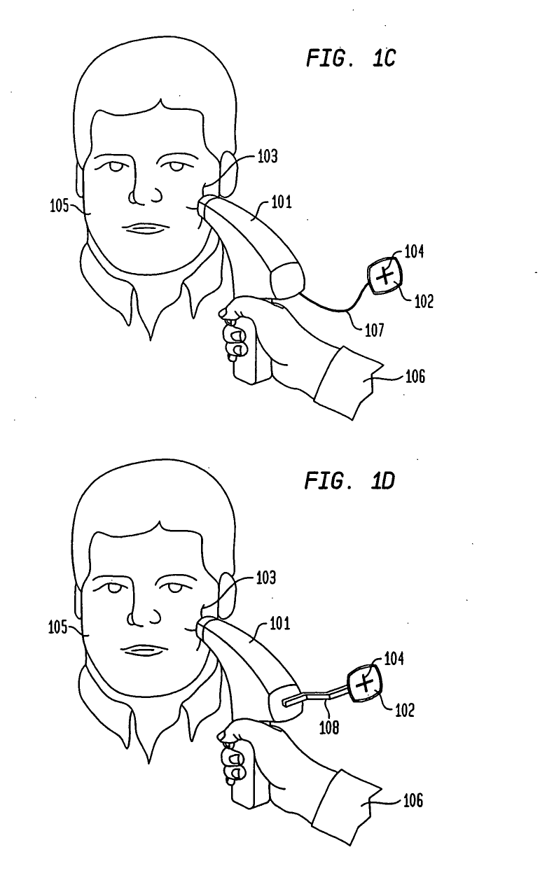

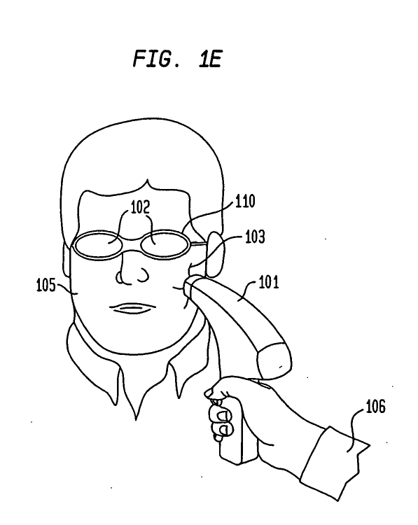

FIG. 1A schematically depicts a handpiece device 112 according to one embodiment of the invention having a housing 101 that includes a handle 114 that allows a user 106, e.g., a medical professional, a home user, or a beautician to hold and aim the device at a selected target treatment area 103. The housing 101, which defines an enclosure in which various components of the device are incorporated, is described in more detail below. The housing 101 can include a head or tip portion 118 at a proximal end that can be placed in proximity to, or in contact with, the treatment area 103 (which can be a surface or subsurface region) of a patient 105 to apply a selected treatment energy (e.g., electromagnetic energy, acoustic, particles, etc.) thereto. The patient 105 may or may not also be the user 106. A display 102 for displaying an image of the treatment area, e.g., at a selected magnification, is coupled to the distal end of the housing 101. The display 102 can be employed to view the treatment area 103, which, in this embodiment, includes two crossing veins, before, during, and after the treatment, as seen in magnified image 104. The treatment area can be located at a depth below the patient's skin surface (including a shallow subsurface region), or can be on the skin surface itself.

Although the display 102 is fixedly attached to the housing in this embodiment, in another embodiment shown schematically in FIG. 1B, the display 102 is moveably mounted to the housing 101 to allow adjustment of its position relative to a viewer 106, e.g., a person applying the treatment, for flexible viewing of the treatment area. In another embodiment shown in FIG. 1C, the display 102 is mechanically and electrically or optically coupled to the housing 112 via one or more flexible wires or optical cable 107, or alternatively, electrically coupled to the housing 112 via wireless, e.g., WiFi connections. In another embodiment shown in FIG. 1D, the display 102 is hingedly attached to the housing 101 via one or more rails 108 or flexible or bendable material for ease of positioning and storage. In some embodiments, the display 102 is removable from the housing 101.

Alternatively, as shown in FIG. 1E, the display 102 can be incorporated in glasses 110 that can be worn by the operator (e.g., medical or other professionals or the patient or customer themselves) to view the treatment area. The glasses can be attached to the housing 101 via a wire, or an optical cable (not shown) or can receive the images or data via a wireless connection. In some embodiments, the operator 105 can use the device to treat himself as shown on FIG. 1E. In some embodiments, two displays are be mounted in the glasses, each corresponding to one eye of the operator. The two displays can be adapted for stereoscopic viewing of the images (three-dimensional vision).

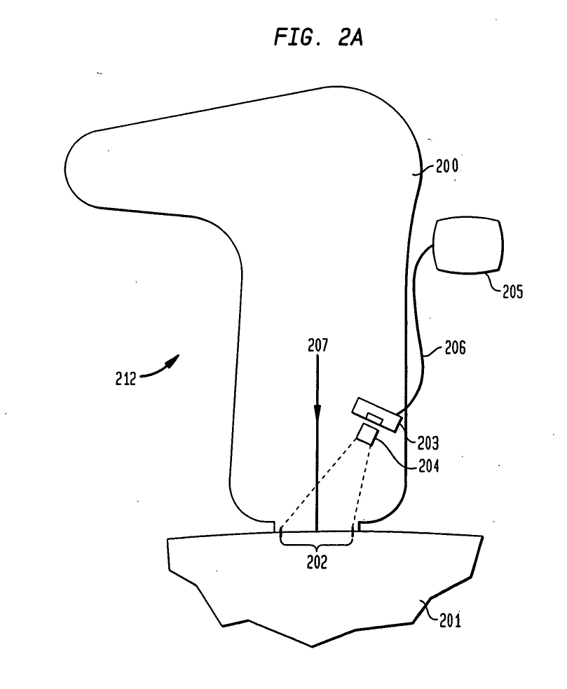

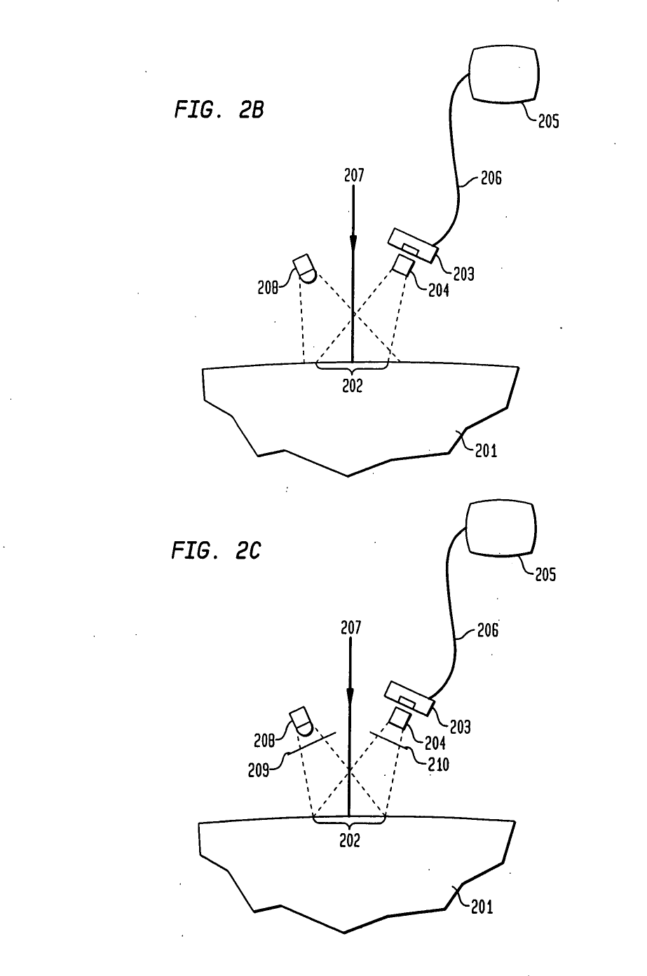

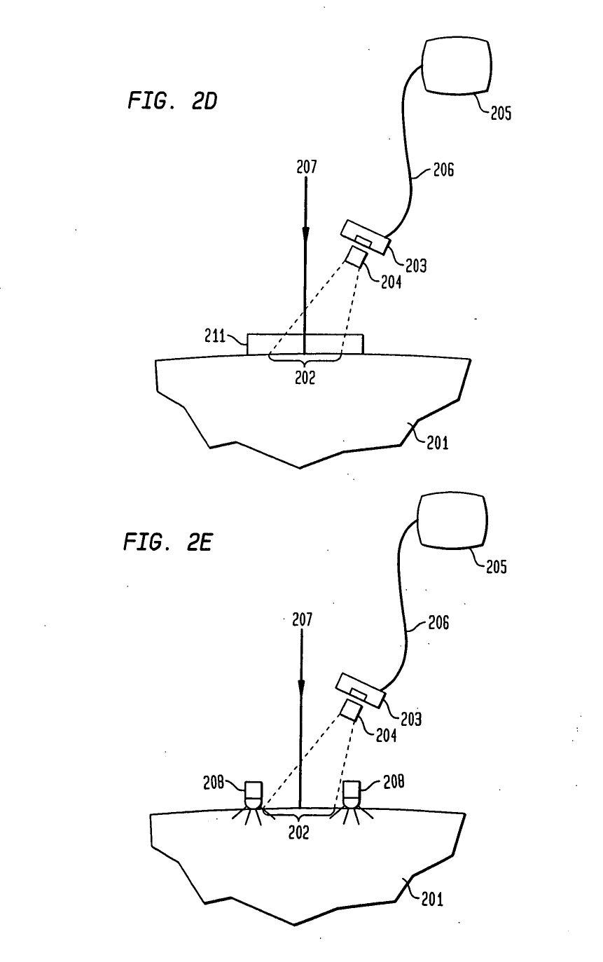

FIG. 2A schematically illustrates that a selected energy flow 207, for example, acoustic, electromagnetic, or kinetic energy, can be directed by a handpiece device 212 according to one embodiment of the invention to a target treatment area 202. A source for generating the applied energy (not shown) can be incorporated in a housing 200 of the device, or alternatively, it can be remotely located relative to the handpiece with its generated energy transmitted via a suitable element, e.g., a waveguide, to the handpiece's housing for delivery to the treatment area. An optical system 204, which can include, e.g., one or more lenses, prisms, mirrors, plates, apertures, masks, filters, phase elements, polarizers, diffractive elements, can direct radiation emanating from a treatment region 201, or a portion thereof, in response to ambient illumination or illumination by one or more radiation sources (not shown) disposed in the housing 200 onto an image capture device 203 that can form an image of the treatment region 201, or a portion thereof (e.g., target treatment area 202). The image capture device 203 can be an analog or a digital device with or without a microprocessor. The image captured by the image capture device 203 can be transmitted via an electrical (, e.g., a cable 206), optical or wireless coupling, or otherwise to a display 205 mounted to the housing for viewing by a user. The image capture device 203, for example, with an integrated microprocessor, display, memory system to store the images, and battery or power supply, can be similar to those used in commercial digital photo or video cameras. The images can be magnified optically or digitally.

With reference to FIG. 2B, in another embodiment, in addition to the components described in FIG. 2A, a handpiece device according to the teachings of the invention can include an illumination source 208, e.g., a light source, for illuminating the treatment area, or a portion thereof, to enhance its imaging by the image capture device 203. The illumination source 208 can be any suitable light generating element, e.g., an LED, a LED array, a lamp, or a laser, having a desired emission spectrum. In some embodiments, radiation from a treatment source can be employed not only for treatment but also for illumination of a target region. Further, in some embodiments, a single source can generate treatment radiation in one wavelength band and illumination radiation in another wavelength band. In some embodiments, the illumination source 208 can be pulsed and/or be synchronized with the image capturing device 203 for improved spatial and thermal resolution. The illumination source 208 can be selected to generate radiation in any desired spectral region. For example, UV, violet, blue, green, yellow light or infrared radiation (e.g., about 290-600 nm, 1400-3000 nm) can be used for visualization of superficial targets, such as vascular and pigment lesions, fine wrinkles, skin texture and pores. Blue, green, yellow, red and near IR light in a range of about 450 to about 1300 nm can be used for visualization of a target at depths up to about 1 millimeter below the skin. Near infrared light in a range of about 800 to about 1400 nm, about 1500 to about 1800 nm or in a range of about 2050 nm to about 2350 nm can be used for visualization of deeper targets (e.g., up to about 3 millimeters beneath the skin surface). Skin infrared emissions can be used for thermal imaging of the skin and/or for control of skin temperature. Although in this exemplary embodiment one illumination source 208 is utilized, it should be understood that in other embodiments the handheld device can incorporate a plurality of such sources, of the same or different emission spectra.

In some embodiments, a variety of optical filters and polarizing elements can be incorporated in a handpiece device of the invention to manipulate, and/or enhance, an image of the treatment area generated by the image capture device. By way of example, FIG. 2C illustrates another exemplary embodiment of a handheld device in which a pair of cross or parallel polarizers (or filters), 209 and 210 are placed, respectively, in front of the illumination source 208 and the optical system 204 to tailor selected parameters, e.g., the polarization and/or the spectrum, of the illumination light and/or the light reflected or emanating from the treatment area in response to the illumination light. For example, a pair of cross polarizers can be employed to suppress reflections from the surface of the treatment region 201 while capturing an image of a target treatment area 202 located at a distance below the skin surface, as described in more detail below.

With reference to FIG. 2D, in another embodiment, a cooling or heating element 211, for example, a sapphire window, can be coupled to the proximal end of the handpiece's housing so that it can be placed in thermal contact with a portion of the patient's skin such as target treatment area 202 during treatment in order to cool or heat the treatment region 201, or a portion thereof such as target treatment area 202, to ensure its temperature remains within an acceptable range. In some embodiments, the element 211 can also enhance imaging of the target by improving the coupling of the illumination light into the skin and coupling the image of the target region into the image capture device. In some embodiments, a layer of a transparent lotion can be placed between the element 211 and the skin to minimize light scattering from the skin surface and reflection from the surface of the element 211 in contact with the lotion layer.

In some embodiments, a handpiece according to the teachings described herein allows preferentially obtaining an image of a portion of a target treatment region that lies a distance below the skin surface. By way of example, in the embodiment shown schematically in FIG. 2E, two or more illumination sources 208, such as, LEDs, lamps, or lasers, that emit radiation in a desired wavelength range, e.g., in a range of about 400 to 1400 nm, or in a range of about 1500 to about 1800 nm, or in a range of about 2050 nm to about 2350 nm, can be placed around a selected target area so as to preferentially illuminate a target region under the skin surface of the target treatment area 202 while minimizing illumination of the skin surface of the target area itself. This approach minimizes light scattering and reflection above the target region (e.g. a lesion), thus enhancing the image contrast of the target region. In some embodiments, light from an illuminator positioned on the head of a user can be used as the illumination light. Skin imaging systems can be built as optical coherent tomography systems or optical confocal microscopy systems to provide images of subsurface targets with very high resolution. In some embodiments, optical registration systems, as discussed below, can be built with decreased spatial resolution to measure average parameters of skin, such as skin pigmentation, skin redness, erythema, and/or skin birefringence.

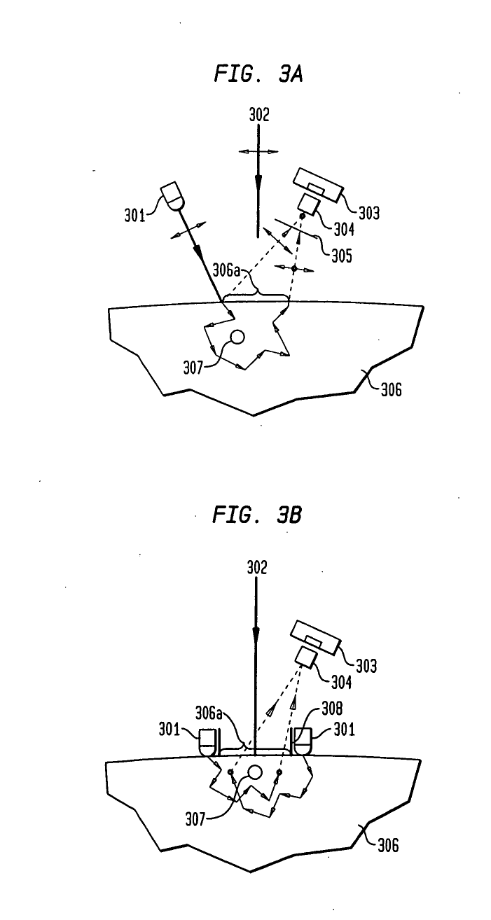

In some embodiments, a handpiece device according to the teachings of the invention is designed to preferentially provide an image of a target region 307 at a depth below the skin surface. For example, FIG. 3A schematically illustrates that an illumination source 301 can generate a linearly polarized beam of radiation to illuminate a target area 306a of a patient's skin 306 (alternatively, or in addition, a treatment beam 302 can be employed for illumination). A portion of the illuminating light generated by the source 301 is reflected by the skin surface of the target area towards a optical system 304 and an image capture device 303, and another portion penetrates into the skin to illuminate a target region 307 located at a depth below the skin surface. A portion of penetrated radiation leaves the patient's skin 306 after undergoing a number of scattering events to reach the optical system 304. While the light reflected from the surface of the patient's skin 306 has substantially the same polarization direction as that of the illuminating light generated by the source 301, the light reaching the optical system 304 after undergoing scattering at a depth beneath the skin can include a significant polarization component in a direction orthogonal to the polarization direction of the illuminating light. In this embodiment, a cross polarizer 305 that substantially blocks light having the same polarization direction as that of the light generated by the source 301 is placed in front of the optical system 304 to prevent the light reflected directly from the skin surface from reaching the optical system 304, and hence the image capture device 303. However, the light rays scattered by the tissue at a selected depth below the skin surface have polarization components that can pass unaffected through the polarizer 305 to be imaged by optical system onto the image capture device 303. The focal plane of the optical system 304 can be adjusted to preferentially image scattered light emanating from a selected target region located at a depth below the skin surface. By way of example, polarized imaging of superficial dermis can be used for diagnostic and control of treatment of collagen using collagen birefringence.

FIG. 3B schematically illustrates another embodiment of a handheld dermatological device described herein that allows preferentially viewing a target treatment region disposed at a depth below the skin surface. Similar to the embodiment shown in FIG. 3A, in this embodiment, a plurality of illumination sources 301 surrounding a target area 306a of a patient's skin surface, below which a target region 307 is disposed, transmit light into the tissue below the skin surface. The transmitted light is scattered by tissue below the skin surface such that a portion of the scattered light illuminates the target region 307. Further, a portion of the light illuminating the target region 307 is reflected/scattered by tissue in the target region and finds its way, e.g., via multiple scattering events, out of the skin in a solid angle directed towards the image capture device. In addition, in this embodiment, an optical shield 308 is disposed between the illumination sources 301 and the portion of the skin surface below which the target region lies so as to reduce, and preferably prevent, illumination of the skin surface by photons emitted by the illumination sources 301. This in turn decreases, and preferably eliminates, reflection of such photons by the target area 306a of the surface of patient's skin 306 onto the image capture device, thereby enhancing the image of the buried target region. The optical shield 308 can be formed of any suitable material that is substantially, and preferably completely, opaque or reflective to photons emitted by the illumination sources 301. Such materials can include, for example, metal, plastic, and glass with special coating. Further, the optical shield 308 can be formed as a single unit surrounding at least a part of the perimeter of the skin surface 306, or alternatively, as a plurality of segments each disposed in proximity of the illumination sources 301 to shield the patient's skin 306 from light emitted by that light source. In this embodiment, the image of the target is formed mostly by photons scattered from the tissue below the target (i.e., by “banana photons,” as discussed in more detail below). This illumination arrangement at the same time minimizes the number of photons scattered from the tissue above the target.

In some embodiments, the optical shield 308 can also function as a mechanism for coupling a current, RF or acoustic energy into the patient's body. For example, the shield 308 can be formed as a plurality of electrodes or transducers that not only prevent photons emitted by the illumination sources 301 from reaching the observation area or optical system 304, but also allow coupling of a current or acoustic energy into the patient's body.

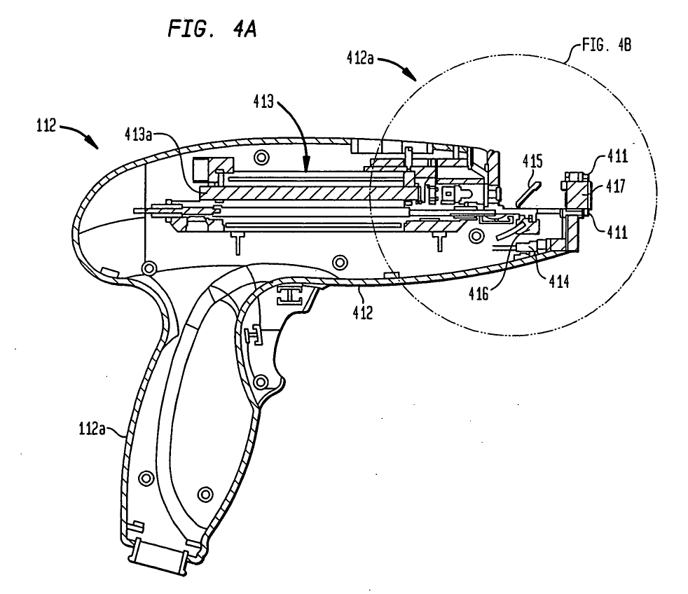

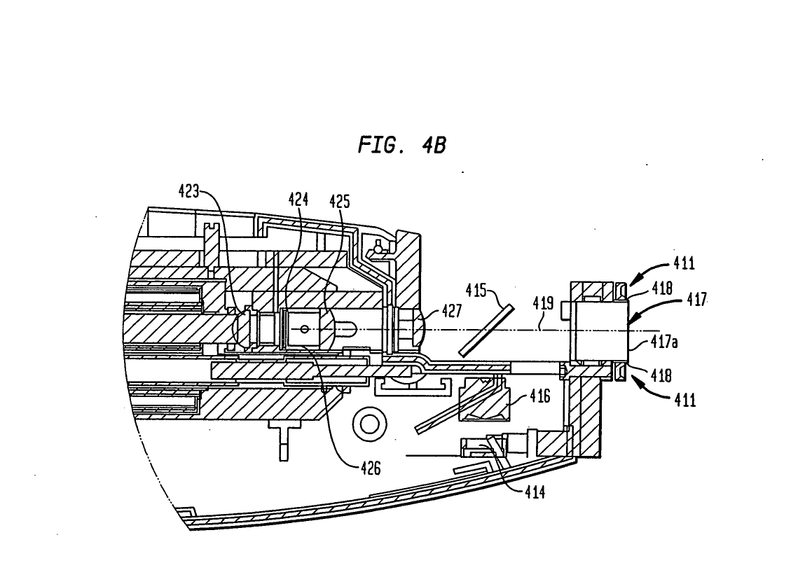

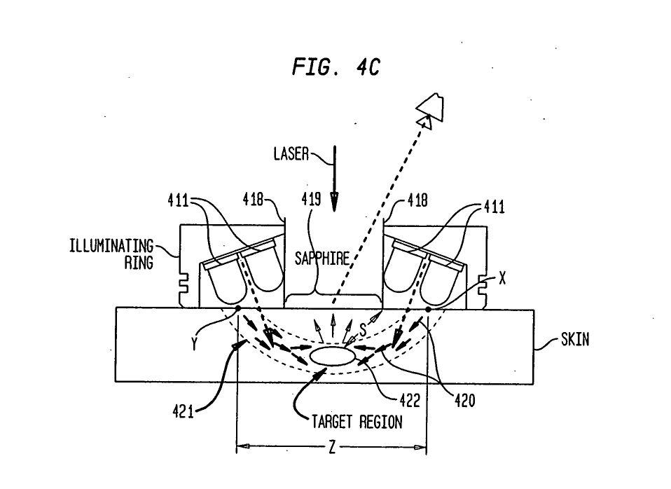

FIGS. 4A, 4B and 4C schematically illustrate an exemplary implementation of the target illumination system depicted in FIG. 3B incorporated in a handheld dermatological device 112 in accordance with one embodiment of the invention. A plurality of illumination sources 411 can be disposed in a head portion 412a of a housing 412 of the device in a ring, quadrant, pentagon, hexagon or any other suitable configuration. The illumination sources 411, herein also referred to as imaging radiation sources, can be utilized to illuminate a target region of a subject's skin located at a depth below the skin surface, as discussed in more detail below. A treatment radiation source 413 disposed in a body portion of the handheld device 112 generates radiation having one or more wavelengths suitable for treating a dermatological condition in the target skin region. In this exemplary embodiment, the treatment source 413 includes a neodymium (Nd) laser generating radiation having a wavelength around 1064 nm. The laser 413 includes a lasing medium 413a, e.g., in this embodiment a neodymium YAG laser rod (a YAG host crystal doped with Nd⁺³ ions, also referred to as an Nd:YAG laser), and associated optics (e.g., mirrors) that are coupled to the laser rod to form an optical cavity for generating lasing radiation. In other embodiments, other laser sources, such as chromium (Cr), Ytterbium (Yt) or diode lasers, or broadband sources, e.g., lamps, can be employed for generating the treatment radiation. By way of example, the device can be employed to treat vascular lesions in depths up to about 2 millimeters with radiation having wavelengths in range of about 400 to about 1200 nm.

In some embodiment, radiation generated by the treatment source 413 can be utilized not only for treating a target region but also for illuminating that region for imaging. For example, the lasing radiation generated by the Nd:YAG laser can be employed for treatment and fluorescence radiation emitted by the laser rod can be utilized for illumination.

The illustrative handheld device 112 further includes an image capture device 414, e.g., a CCD camera, for generating an image of a target region of the subject's skin. More particularly, as discussed in more detail below, radiation reflected from a skin target region can be directed by a beam splitter 415 to a lens 416 that in turn focuses the radiation onto the image capture device 414.

A sapphire window 417 mounted at the tip of the head portion allows extracting heat from a portion of the skin surface that can be in thermal contact therewith before, during or after application of treatment radiation.

Referring to FIG. 4B, shield 418 is mounted in the head portion 412a between the sapphire window 417 and the illumination sources 411 so as to inhibit, and preferably prevent, radiation generated by the sources 411 from reaching a surface of a skin segment that will be in contact with a surface 417a of the window formed of sapphire or other transparent thermo conductive material, when the device is utilized for imaging and/or treating a target skin portion, as discussed in more detail below. In other words, the shield prevents radiation from the illumination sources 411 from intersecting a portion of an optical path 419 (through which treatment radiation from treatment radiation source 413 can be transmitted to a target region and through which radiation emanating from the target region, e.g., in response to illumination by illuminating sources 411, can reach the image capture device 414) that extends through the sapphire window 417. The shield 418 is preferably formed of a material that is opaque to the radiation wavelengths generated by the illumination sources 411. Some examples of materials from which the shield 418 can be formed include, without limitation, glass, metal or plastic. In some embodiments, the internal shield surface can be coated with a material that is highly reflective to the treatment radiation to minimize heating by the treatment light, and hence minimize potential skin damage due to such heating. In addition, the reflective coating can improve the treatment efficiency by providing a photon recycling effect.

With reference to FIG. 4C, in use, the handheld device can be manually manipulated, e.g., by utilizing a handle 112a thereof (FIG. 4A), so as to place its head portion in proximity of a subject's skin surface such that the surface 417a of the sapphire window 417 is in thermal contact with a segment 430 of the skin surface. The illumination sources 411 can be activated to generate radiation that penetrates the skin surface while the shield 418 prevents this radiation from illuminating the surface of skin segment 430. As shown schematically by arrows 420, the radiation penetrating the skin is scattered by the skin tissue to illuminate a curved skin segment 421 in a portion of which a target skin region 422 is located. Due to the curved profile of the skin segment 421, the photons from the illumination sources 411 that illuminate skin segment 430 via scattering by skin tissue are herein referred to as “banana photons.” In other words, the term “banana photons” refers to those photons that propagate from one point on the skin surface (e.g., point X) to another point on the skin surface (e.g., point Y), which are separated from one another by a distance Z. The configuration of the light field generated by the banana photons is similar in shape to a banana with one end at X and the other at Y. The penetration depth of the banana photons depends on the radiation wavelength and the distance Z. A maximum penetration depth is roughly about 0.5Z. A distance S between the treatment target and the shield or the place of coupling of the illumination light into the skin can control the penetration depth of the banana photons. Deeper targets need a larger distance S. In general, the distance S is chosen to be larger than h (S>h), wherein h is a maximum depth of the target. The sources 411 can be direct, such as LEDs, diode lasers or lamps with prelensing (as shown), or indirect such as the same sources whose light is coupled into waveguides (e.g., fibers) for directing light to the skin. More particularly, the output ends of the lens or waveguides can be optically attached to the skin for better coupling of the illumination light into the skin. In some embodiments, by direct coupling of illumination light into the skin, the shield 418 can be eliminated.

A portion of the “banana photons” illuminating the target region 422 are reflected or scattered by the skin tissue in the target region 422 into a solid angle extending to the skin surface segment 419. In other words, a portion of the “banana photons” are scattered by tissue in the target region, mostly from below the target, so as to exit the skin via the skin surface segment 430, which is shielded from direct illumination by illumination sources 411. Referring back to FIG. 4B, the beam splitter 415 directs this radiation towards the image capture device 414, via the lens 416, while allowing the treatment radiation generated by the treatment radiation source 413 to pass through and reach the skin segment 422 (FIG. 4C) via the sapphire window 417. The treatment radiation can penetrate the skin to treat a dermatological condition present in the target region. The fluorescence light from the laser rod or simmer mode light from a lamp can be used for illumination. In addition, the treatment light itself can be employed for illumination to provide, for example, a better resolution of the target coagulation process during a treatment pulse.

As noted above, the shield 418 prevents the radiation generated by the illumination sources 411 from illuminating the skin surface segment 430 so as to avoid reflection of this radiation from the skin surface onto the image capture device 414, thereby maximizing the signal-to-noise ratio of the image of the target region formed by the image capture device through detection of a portion of the “banana photons” scattered by the target region.

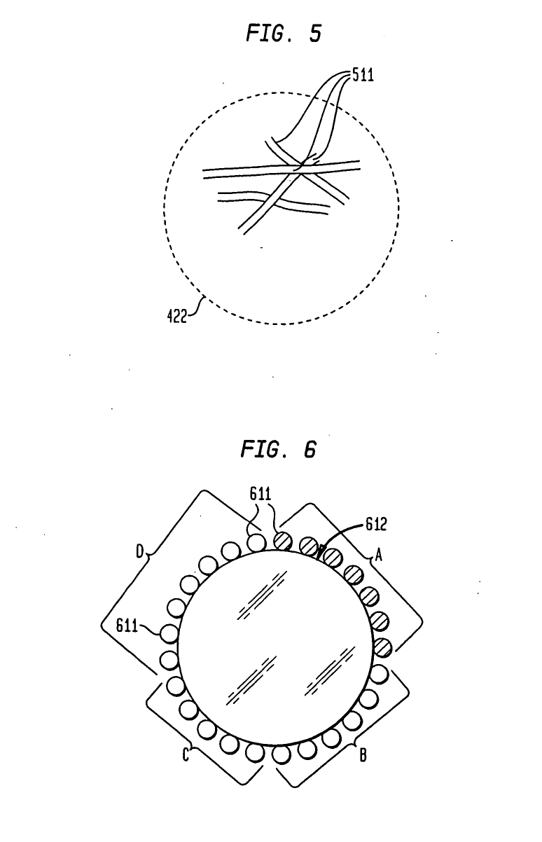

The image of the target region 422 can be utilized by an operator, e.g., a medical professional, to select a portion of the target region, or the entire target region for treatment. For example, FIG. 5 shows a schematic image of the target region illustrating a plurality of vessels 511, removal of one or more of which may be desired.

A plurality of images can be obtained during application of treatment radiation to assess the progression of the applied treatment in real-time. Further, such images can indicate when the application of the treatment radiation should be terminated. Alternatively, subsequent to treating a target region, one or more images of that region can be obtained to determine if the applied treatment was successful. For example, a color change exhibited by a vessel under treatment can indicate whether that vessel has been coagulated in response to treatment radiation. The images can be presented to a user via a display (not shown) mounted to the housing in a manner described in connection with the above embodiments. Further, one or more images of vessels can be used to control the pressure by which the handheld device is pressed against the skin. By controlling the pressure, blood can be removed from or pumped into certain portions of the vessels to provide control of the treated vessel, thereby enhancing the treatment efficiency and preventing over-treatment. For example, for highly dense spider veins, the blood volume within the veins can be minimized before application of a treatment pulse by applying a positive pressure to prevent side effects. The treatment can be repeated several times in the same area with different pressures. Using a negative pressure, it is possible to increase the blood volume within vessels before treatment. Hence, the described image techniques can be utilized to control treatment results. Moreover, heating of the blood can result in transformation of oxyhemoglobin into other forms that exhibit different absorption spectra (e.g., methemoglobin). Thus, utilizing broad spectrum sources or multiwavelength sources, the temperature transformation of blood can be detected. For example, green LEDs (490-560 μm) can be used for visualization of vessels, such as leg veins or facial spider veins, before treatment, while red and infrared (IR) LEDs (600-670 nm, 900-1200 nm) can be used for visualization of heated blood. LED illumination in a range of about 670 nm to about 750 nm can be used to distinguish blood vessels and veins with different oxyhemoglobin concentrations. Further, coagulation of vessels can be detected through the loss of image of the vessels due to stoppage of blood supply through the vessels or high scattering by the coagulated tissue.

Referring again to FIGS. 4A and 4B, the exemplary handheld device 112 includes a zoom assembly comprising three lenses 423, 424 and 425. The lens 425 can move axially (i.e., along a direction of propagation of the treatment beam) within a slider element 426 relative to the lenses 423 and 424 so as to change the cross-sectional diameter of the treatment beam. By way of example, the cross-sectional diameter of the treatment beam can vary in a range of about 1 mm to about 15 mm.

In addition, in this exemplary embodiment, a snap-in lens 427 can be employed to augment the zoom assembly and/or to modify the cross-sectional shape of the treatment beam. For example, the lens 427 can be a cylindrical lens to impart an elliptical cross-sectional shape to the treatment beam. Other lens types can also be employed.

In some embodiments, the image capture device 414 can be a video camera for generating a movie that can show, for example, a temporal progression of an applied treatment. Providing visualization techniques in combination with treatment energy in one single device affords a user the opportunity to control a number of treatment pulses in a pulse stacking mode. For example, the device can deliver energy to a target region every 1 second (stacking mode) until coagulation of the target is completed. At this point, the user can interrupt firing of the pulses.

In some embodiments, the illumination sources mounted in the head portion of the handheld device can provide radiation in different spectral ranges (e.g., different colors) for illuminating a target region. For example, FIG. 6 schematically depicts a plurality of illumination sources 611 mounted at a tip of a handheld device according to one embodiment of the invention and a shield 612 that prevents radiation generated by these sources from illuminating a selected skin surface segment through which an image of a target region illuminated by these sources can be obtained, in a manner described above. In this exemplary embodiment, the radiation sources in each of quadrants A, B, C and D generate radiation having one or more wavelengths different than those generated by the sources in the other quadrants. For example, while the sources in the quadrant A can provide red light, the sources in the quadrant B can generate blue light. The radiation sources in different quadrants can be activated concurrently or in succession, or in any other desired temporal pattern, to illuminate a target region. For example, the target region can be illuminated simultaneously with two or more different radiation wavelengths (e.g., two different colors). Alternatively, the target region can be illuminated by sources generating radiation with the same spectral components at a time (e.g., one color at a time). In this manner, images of the target region illuminated by different radiation wavelengths can be obtained. In some embodiments, one or more of the illumination sources can generate radiation in two or more wavelength bands.

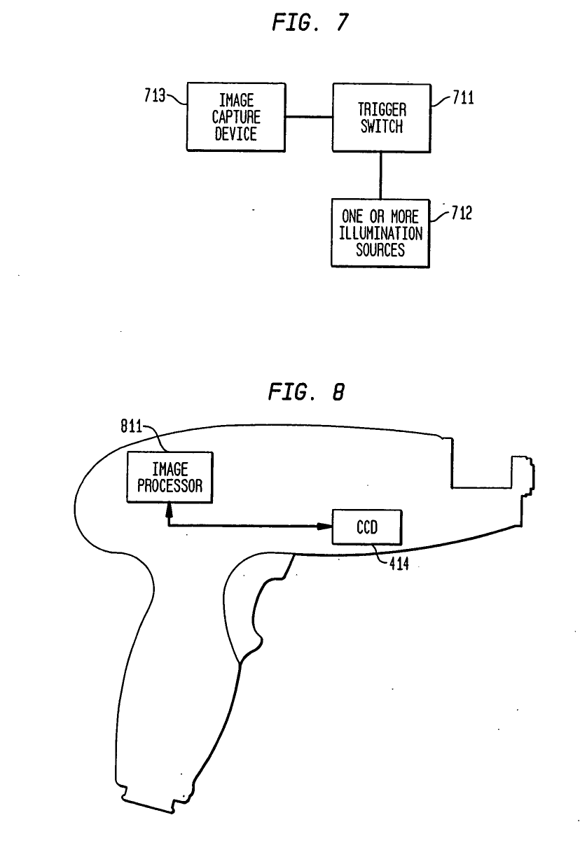

In some embodiments, the image capture device can be activated in synchrony with activation of one or more radiation sources utilized for illuminating a skin target region. By way of example, the image capture device can be activated to acquire an image of the target region each time the illumination sources in one of the quadrants (FIG. 6) are triggered. For example, with reference to FIG. 7, the handheld device can include a control unit (e.g., a triggering switch) 711 for sending concurrent triggering signals to selected ones of the illumination sources 712 mounted on the device and an image capture device 713. Alternatively, one triggering signal can be delayed relative to another by a selected time duration. For example, the triggering signal activating the image capture device can be delayed relative to that activating one or more of the radiation sources.

With reference to FIG. 8, in some embodiments, the processing of the images can be achieved by a microprocessor 811 incorporated in the handheld device that is in communication with the image capture device 414.

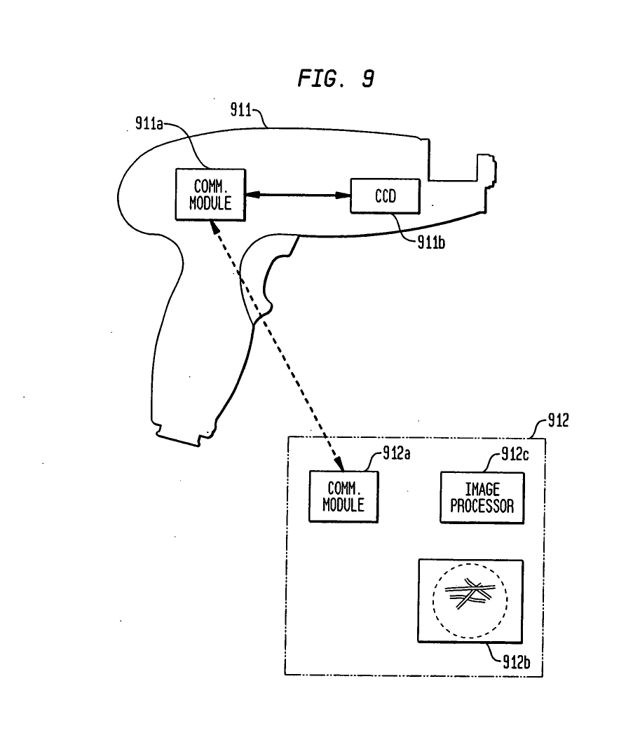

Alternatively, with reference to FIG. 9, the images or data can be transferred from a handheld device 911 according to one embodiment of the invention to a separate computing device 912 on which appropriate software for image construction can be executed. For example, in some embodiments, images of a target skin region acquired by the handheld device can be transmitted by employing, for example, a wireless protocol to the computing device 912, which can be remotely located relative to the handheld device. For example, the handheld device can include a communications module 911a for transmitting images or data acquired by an image capture device 911b to the computing device 912, via a corresponding communication module 912a of the computing device. The computing device 912 can include a display 912b for displaying the images to a user, e.g., a medical professional. Further, the computing device 912 can optionally include an image processing module 912c for processing the images or data of the target region.

In some embodiments, the image of the target region can be analyzed by employing image recognition techniques to extract selected features, e.g., vascular legions. These extracted features can be displayed on a display mounted to the handheld device, such as a display similar to that shown above in FIG. 1A in connection with the handheld device 101.

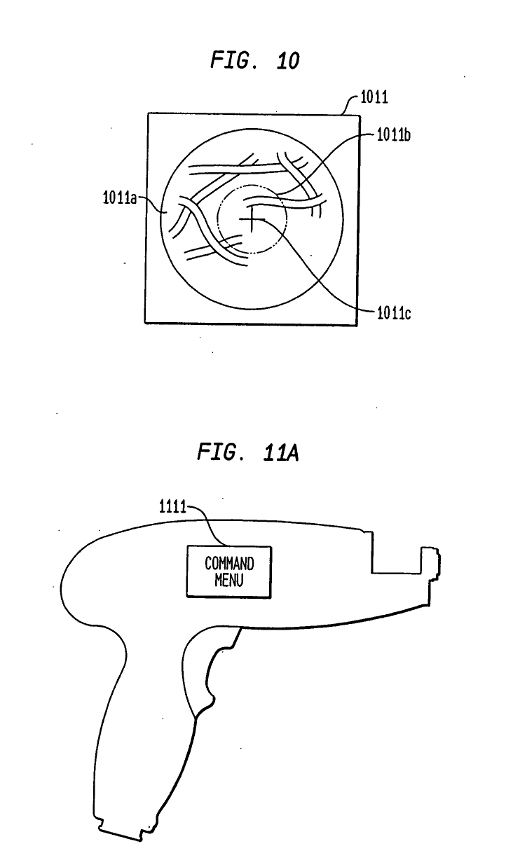

With reference to FIG. 10, in some embodiments, a display unit 1011 of a handheld device according to one embodiment of the invention can present not only an image 1011a of a target skin region, but also a graphical element 1011b, e.g., a circle, that schematically depicts the cross-section of the treatment beam relative to the target region. In some embodiments, the user can select the portion of the target region identified by the graphical element 1011b, e.g., the portion circumscribed by the circle, for magnified viewing. For example, with reference to FIG. 11A, the handheld device can provide a user interface, for example menu 1111, to a user in a portion of the display utilized for displaying images, or in a separate display, that can be navigated to select commands for controlling selected display characteristics of the image of the target region. For example, the menu can provide commands for magnifying the portions of the image associated with a portion of the target region to which treatment radiation is being applied. Such magnification can be achieved, for example, automatically in response to the user's selection by sending appropriate signals to a zoom lens system of the handheld device, such as the zoom lens assembly shown in the above handheld device 112 (FIG. 4A). For example, a piezoelectric element electrically coupled to a movable lens of a zoom lens assembly can be activated in response to the user's selection to move that lens, thereby modifying the magnification of the displayed image of the target region.

The graphical elements suitable for displaying the position of a treatment beam relative to an image of a target region are not limited to those described above. For example, referring again to FIG. 10, a cross-hair 1011c can be employed to denote the center of the treatment beam's cross section. Such visual aids facilitate positioning of the handheld device relative to a patient's skin to more effectively apply treatment radiation to a portion of the target region whose image is displayed. These alignment features can significantly increase efficacy and safety of the treatment. For example, in the absence of such features, it is difficult to position small treatment beams (e.g., spot size less than 3 mm) on small treatment targets, such as vessels.

With reference to FIG. 11B, a handheld device 1112, according to one embodiment of the invention, can include a microprocessor 1113 electrically, optically, or via a wireless connection, coupled to an image capture device 1114 to receive images or data acquired by the image capture device. The microprocessor can utilize these images or data to monitor an applied treatment. For example, the microprocessor can be programmed to compare changes in selected parameters of the skin tissue (e.g., color of a vessel) extracted from the acquired images with threshold values for these parameters stored, for example, in a memory element containing a database 1115. The database can be maintained in the handheld device, or alternatively, the needed data can be downloaded to the device from a remote database. By way of example, comparison of color of a vessel, a pigment lesion, or a tattoo, irradiated to cause its coagulation, can signal that the treatment has been successful. Upon detecting threshold values for one or more selected parameters, the microprocessor can alert a user, e.g., by providing a visual, audible, or other signal, that the parameters have reached the preset threshold values. The threshold values can signal, for example, completion of a treatment protocol, or onset of an undesirable condition, e.g., the temperature of skin exceeding a threshold value.

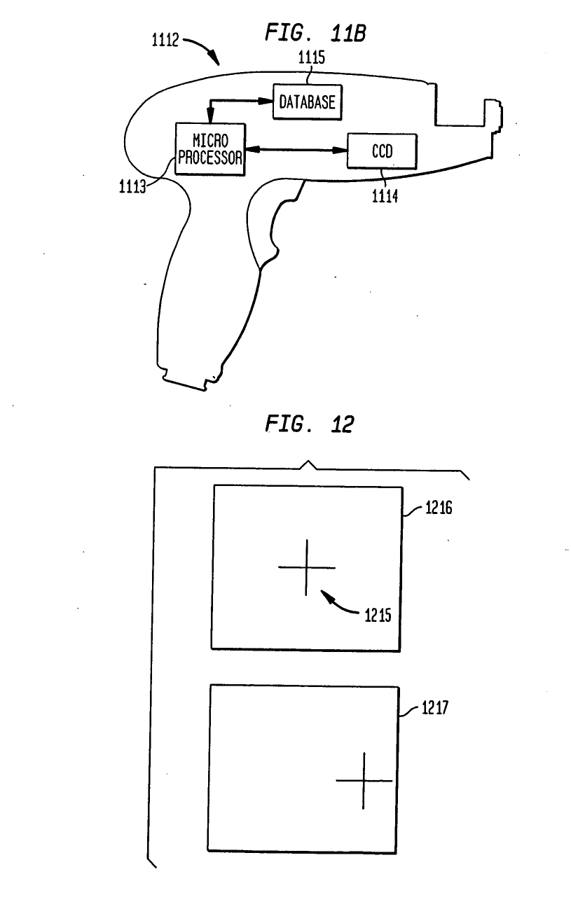

In some embodiments, the handheld device can track the position of a target region, e.g., a treatment site, which can be identified by a marker in an image, from one image to the next. For example, with reference to FIG. 12, a marker 1215 is provided on an image 1216 of a target skin region to identify a selected site, e.g., a treatment site. A subsequent image 1217 may obtained such that it is shifted relative to the image 1216 (for example, a result of movement of the handheld device). In these embodiments, the position of the marker is tracked such that it can be presented at the appropriate location of the image 1217 identifying the selected site in the new image. Such tracking can be particularly advantageous when one image is shifted relative to a subsequent image, for example, as a result of motion of the handheld device. More specifically, in some embodiments, the microprocessor can implement an algorithm by which a marker placed on one image to identify a selected site (e.g., the treatment site) is transferred to a subsequent image while taking in account the motion of the image capture device between acquisition of the two images.

In one exemplary tracking algorithm, the motion of an image pixel can be modeled as a combination of translation in the image plane (herein referred to as x-y plane) and rotation about an axis orthogonal to this plane. The following notations are employed in describing the algorithm: x, y denote a pixel coordinates, Vx, Vy velocity components of a pixel along the x and y coordinates; Ux, Uy indicate components of translation velocity (the same for all pixels in each image but may vary from one image to another); Rx, Ry denote the coordinates of the center of rotation at which the rotation axis cross the x-y plane (all pixels in each image rotate around the same center but the center may vary from one image to another); and c denotes the angular velocity of rotation. An optical flow model of the pixels can then be described by the following relations:
Vx=Ux−ω·(y−Ry),
Vy=Uy+ω·(x−Rx). (1)

The above equations can be cast in a linear format by introducing variables X₁, X₂ and X₃ defined as follows:
X₁=Ux+ω·Ry, X₂=Uy−ω·Rx, X₃=ω. (2)
More specifically equations (1) take the following form when the variables X₁, X₂ and X₃ are employed:
Vx=X₁−y·X₃,
Vy=X₂+x·X₃. (3)

The following optical flow constraint equation can be utilized to determine the change in the position of a pixel between images:

$$Vx \cdot \frac{\partial I}{\partial x} + Vy \cdot \frac{\partial I}{\partial y} = - \frac{\partial I}{\partial t} . \qquad ( 4 )$$

wherein I(x,y,t) represents the pixel brightness at a location (x,y) in an image at a time t. Utilizing the notations Ix, Iy and It for the derivatives in equation (4) and substituting values for Vx and Vy defined by equations (3) into equation 4, the following equation is obtained:
Ix·X₁+Iy·X₂+(x·Iy−y·Ix)·X₃=−It. (5)

The above equation (5) should be valid for every pixel of an image. When a region of the image represented by several pixels is selected, a system of equations can be obtained, which can be defined as follows:
Ixₖ·X₁+Iyₖ·X₂+(xₖ·Iyₖ−yₖ·Ixₖ)·X₃=−Itₖ, (6)

wherein the index k=1, 2, . . . n can represent the pixel number.

The coefficients of X₁, X₂ and X₃ in the above set of equations can be represented by the following matrix:

$$A = [ \begin{matrix}{Ix}_{1} & {Iy}_{1} & {x}_{1} \cdot {Iy}_{1} - {y}_{1} \cdot {Ix}_{1} \\ {Ix}_{2} & {Iy}_{2} & {x}_{2} \cdot {Iy}_{2} - {y}_{2} \cdot {Ix}_{2} \\ ⋯ & ⋯ & ⋯\end{matrix} ] . \qquad ( 7 )$$

By way of example, if the number of pixels (n) is selected to be three (n=3), then A is a square matrix, and the values of X₁, X₂ and X₃ can be obtained by utilizing the following relation:

$$[ \begin{matrix}{X}_{1} \\ {X}_{2} \\ {X}_{3}\end{matrix} ] = - {A}^{- 1} \cdot [ \begin{matrix}{It}_{1} \\ {It}_{2} \\ {It}_{3}\end{matrix} ] . \qquad ( 8 )$$

where A⁻¹ is the inverse of the matrix A.

In many embodiments of the invention, the number of pixels is chosen to be much larger than 3 so that the matrix A is not square and the system of equations (6) is redundant. Utilizing the least square criteria, the following solution can be obtained:

$$[ \begin{matrix}{X}_{1} \\ {X}_{2} \\ {X}_{3}\end{matrix} ] = - {( {A}^{T} \cdot A )}^{- 1} \cdot {A}^{T} \cdot [ \begin{matrix}{It}_{1} \\ ⋯ \\ {It}_{n}\end{matrix} ] . \qquad ( 9 )$$

The algorithm for tracking a marker initially positioned at xₘ,yₘ in a first image can then include the steps of choosing a number of pixels (preferably larger than 3) in a central portion of the first image and evaluating derivatives Ixₖ, Iyₖ at these pixels to generate the matrix A. In many embodiments, the determinant of the matrix A^{T}A is calculated to ensure that it is not too small. If the determinant is too small, additional pixels can be selected and the matrix A regenerated. Using the first image and a second image, the time derivative Itₖ are evaluated at the selected pixels (e.g., by assuming dt=1). The above relation (9) is then employed to evaluate X₁, X₂ and X₃. The values of Vx and Vy are evaluated at the marker position (x=xₘ, y=yₘ). The marker position in the second image can then be determined as follows:

xₘ→xₘ−Vx·dt, yₘ→yₘ−Vy·dt; dt=1. The above steps can be repeated for the subsequent images.

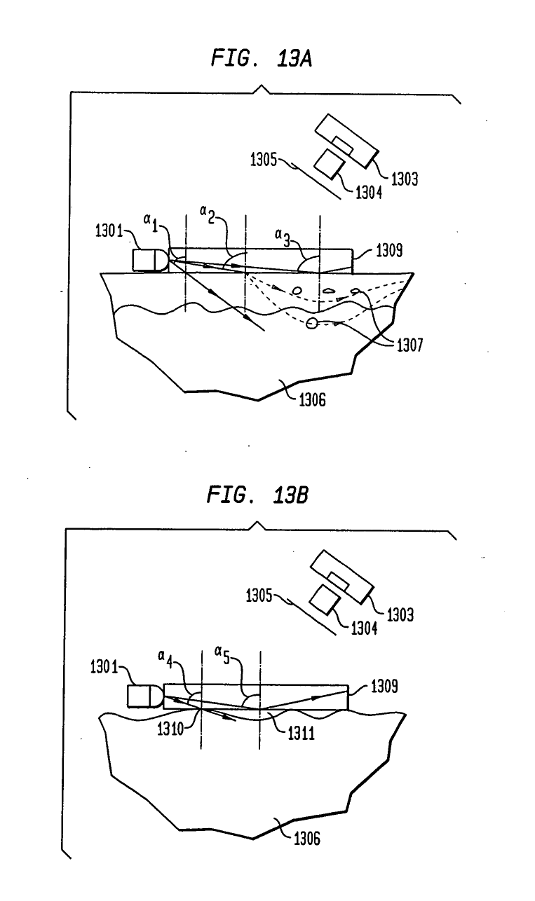

With reference to FIG. 13A, in another embodiment, refractive illumination waves, or evanescent waves, traveling at an interface of a coupling element 1309 and the surface of an observation area of a patient's skin can be employed to illuminate a skin surface or a thin subsurface layer of the skin for imaging thereof by an image capture device 1303. This embodiment can be used for precise imaging and control of skin surface conditions, for example, stratum corneum structure, pore size, sebaceous follicle opening, hair follicle opening, skin texture, wrinkles, psoriasis. More particularly, the coupling element 1309, which is disposed over the observation area, is selected to be substantially transparent to radiation emitted by the illumination source 1301, which is optically coupled to the coupling element 1309. By control of refractive index of the guiding element and incident angle of the illumination radiation at the skin contact surface of the guiding element, imaging contrast of a visualized target can be enhanced. The illumination source 1301 can illuminate the optical coupling element 1309 from a side surface thereof. A portion of the light entering the coupling element 1309 is either totally internally reflected at the interface of the optical element and the patient's skin, or partly penetrates into the skin at a control angle while generating refractive coupled illumination light waves, traveling along the interface as surface or waveguide electromagnetic waves that penetrate to a certain depth of the patient's skin. Such refractive coupled illumination waves can illuminate a subsurface region of the patient's skin, e.g., stratum corneum, epidermis or a top portion of the dermis up to 300 microns depth. Depth of penetration into the skin depends on the illumination wavelength, angle of incidence αᵢ, the refractive index of the coupling element 1309 (n₁) and effective refractive indices of skin layers, such as stratum corneum (n₂), epidermis (n₃), upper dermis (n₄) and deep dermis (n₅), where n₂>n₃>n₄>n₅. A portion of the refractively coupled illumination light is scattered by the target 1307. The scattered light can then be focused by the optical system 1304 onto the image capture device 1303 to generate an image of the subsurface region. The contrast of the target image is maximized when the skin structures below the target cause minimal scattering light noise. The refractive coupling of the illumination wave can be optimized for different depths of penetration or for maximum skin resolution. For example, if an incident angle (α₁) is less than arcsin(n₂/n₁) (α₁<arcsin(n₂/n₁)), the illumination light can penetrate into the skin.

If arcsin(n₃/n₁)<α₂<arcsin(n₂/n₁), the illumination wave propagates mostly into stratum coreum. If arcsin(n₄/n₁)<α₂<arcsin(n₂/n₁), the illumination wave propagates mostly into epidermis and stratum corneum. If arcsin(n₅/n₁)<α₂<arcsin(n₂/n₁), the illumination wave propagate mostly into upper dermis, epidermis and stratum corneum. These conditions are applicable for wavelengths with low absorption and scattering in the skin bulk (500-1400 nm, 1500-1800 μm). If α₃>arcsin(n₂/n₁), the illuminating light totally internally reflects from contact surface of coupling element 1309. In this case, an image on imaging capture device 1303 looks like uniform field and can not be used for subsurface target visualization. However, this condition can be very effective for obtaining high contrast image of skin surface. For example, with reference to FIG. 13B, the total internal reflection mode can be used for visualization of skin surface irregularities, holes in stratum corneum, distribution on the skin surface of sebum, bacteria, water, oil, pores, glands and follicles opening. If α₄<arcsin(n₂/n₁), the illumination light penetrate into the skin and this contact area 1310 is imaged on image capture device 1303 as a bright or a black spot, depending on the initial adjustment of the image capture device. But if arcsin(n₆/n₁)<α, where n₆ is reactive index of air in the gap 1311 or lotion which fills this gap, the illumination light totally reflects from the contact surface and gap 1311 is invisible to the image capture device 1303. As a result, a skin texture image can be acquired by the image capture device 1303.

In other embodiments, the total internal reflection from the surface between the coupling element 1309 and the skin can be interrupted by a material on the skin having a high absorption coefficient at the illumination wavelengths. For example, for detection of water distribution on the skin surface, radiation with wavelengths around 1450, 1900 or 2940 nm can be used. Further, wavelengths corresponding to the peaks of lipid absorption can be employed for visualization of oil or sebum distribution on the skin. By way of example, this embodiment can be used for control of topical drug or lotion distribution on the skin.

Further, a lotion (not shown) can be applied to the skin surface 1306 below the coupling element 1309. The lotion's refractive index can be selected to adjust the penetration depth of photons illuminating the subsurface region, thereby controlling the depth of observation. The use of refractively coupled illumination waves for imaging of shallow subsurface regions of a patient's skin can provide certain advantages. For example, the evanescent waves, which exponentially decay with the depth of the skin, can effectively illuminate a selected subsurface region of interest and not deeper regions. This selective illumination advantageously enhances signal-to-noise ratio of an image generated by capturing photons reflected from the skin in response to illumination.

The above exemplary system in which refractively coupled illumination waves are employed to image subsurface skin regions can be incorporated in a handheld device according to one embodiment of the invention. In some cases, the optical coupling element 1309, in addition to facilitating generation of illumination subsurface waves, or evanescent waves, can also extract heat from the skin portion with which it is in thermal contact.

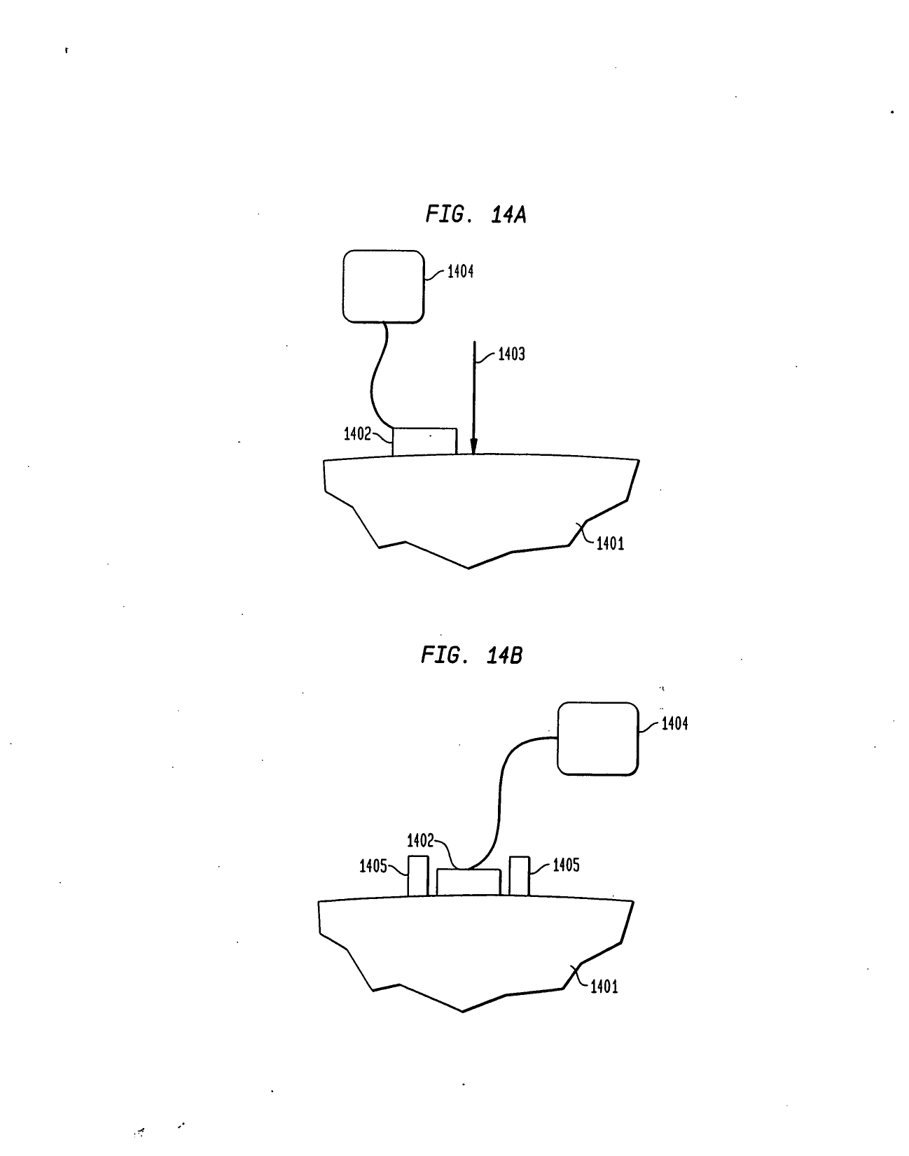

With reference to FIG. 14A, in some embodiments, the handheld device can include an array of capacitive, piezo and/or optical sensors 1402 that can be coupled to a target treatment area to provide information regarding selected properties thereof. For example, an array of capacitive sensors can be employed to generate a dielectric image of the treatment area before, during and/or after irradiation of the target area of the patient's body 1401 by a beam 1403 of electromagnetic radiation or any other suitable energy source. For example, capacitive touch sensors marketed by Orient Drive, Inc. of Mountain View, Calif. under the trade designation MMF200-OD1-01 can be utilized for this purpose. This sensor is marketed as an integrated ASIC having a processor as well as SRAM and flash memory. Other sensors that do not include integrated processor and/or memory can also be utilized in the practice of the invention. The resolution of the sensors can be selected to be sufficiently high to distinguish a treatment target, e.g., a vein, from its surrounding area. The data obtained by the sensor array can be transmitted to a display 1404 mounted to the device's housing for presentation to a user in a selected format. For example, the display can present dielectric data as a false color image in which each color hue represents a measured value of dielectric constant.

With reference to FIG. 14B, in another embodiment, a diagnostic/therapeutic dermatological handheld device according to the teachings of the invention can include, in addition to an array of capacitive, piezo or optical sensors 1402 coupled to a display, a plurality of electrodes or transducers 1405 that can be disposed in proximity of a selected target area so as to couple an electrical current or acoustic energy into the target are of the patient's body 1401. In this embodiment, optical sensor 1402 can be built as a confocal microscope or an optical coherent tomography head.

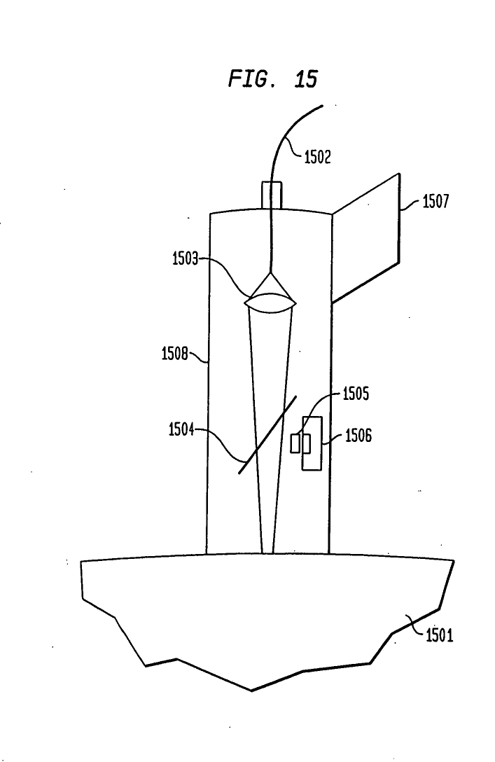

FIG. 15 schematically depicts a cross-sectional view of a handpiece dermatological device according to the teachings of the invention that includes a housing 1508 into which a waveguide 1502, for example, an optical fiber, is coupled to transmit energy, e.g., electromagnetic energy, from a source (not shown), e.g., a source remotely located from the device, to the handpiece for delivery onto a treatment area 1501. In this embodiment, the waveguide 1502 is an optical fiber that is optically coupled to a lens that focuses light delivered by the fiber onto a selected treatment area. A beam splitter 1504 allows the light directed by the lens 1503 towards the treatment area 1501 to pass through while it diverts a portion of light reflected from the treatment area, either in response to illumination by the treatment beam or ambient illumination, or in response to illumination by a separate light source, to an image capture device 1506 via an optical system 1505. The image capture device 1506 in turn generates an image of the treatment area, or a portion thereof, and transmits the image to a display 1507 for viewing by a user.

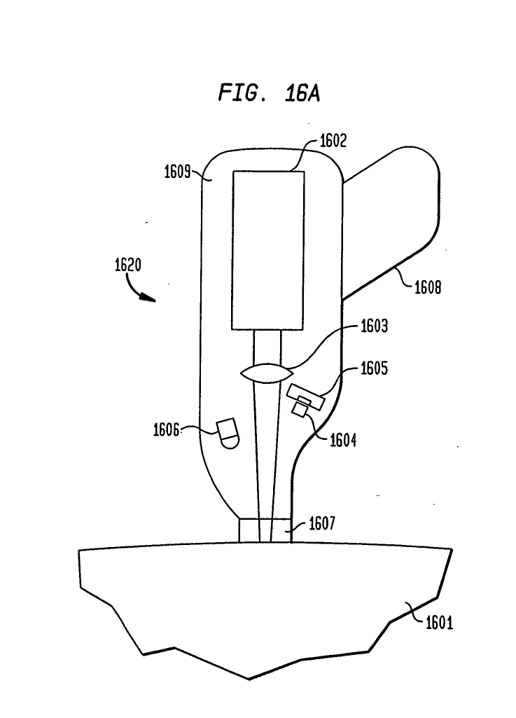

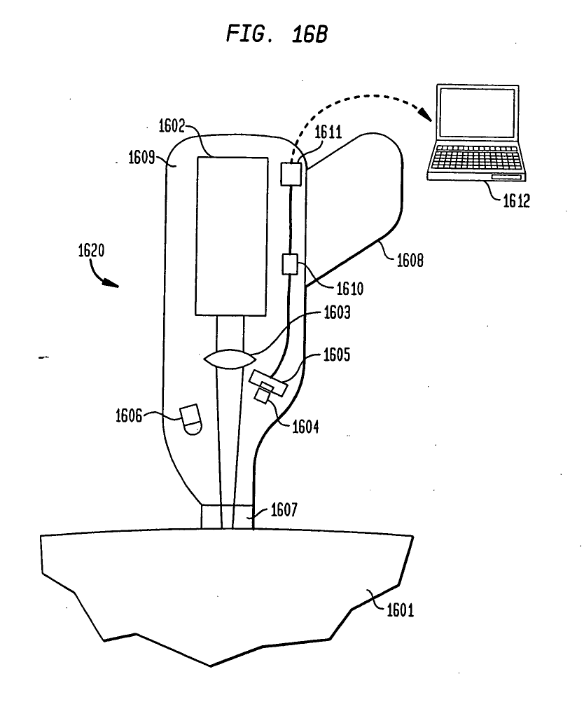

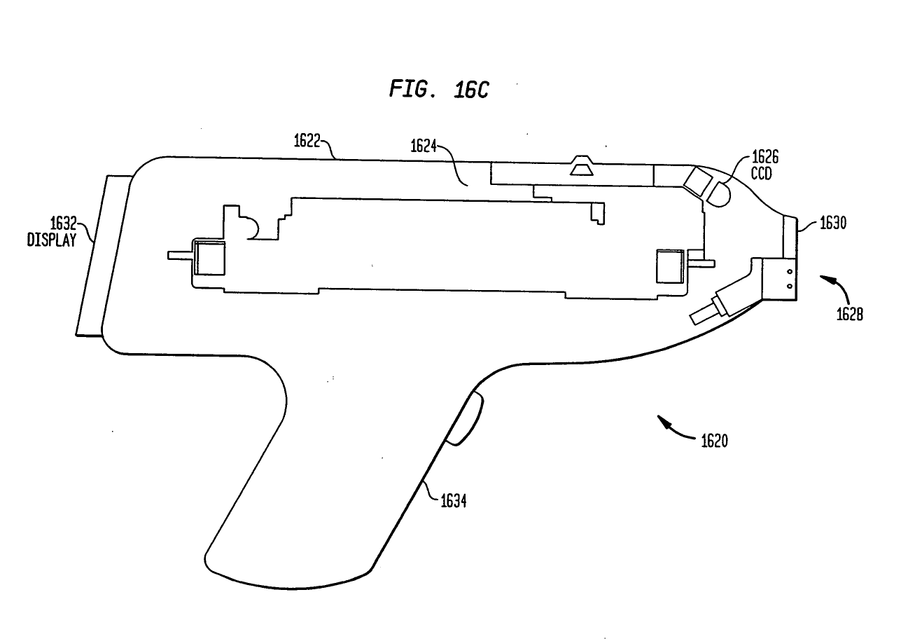

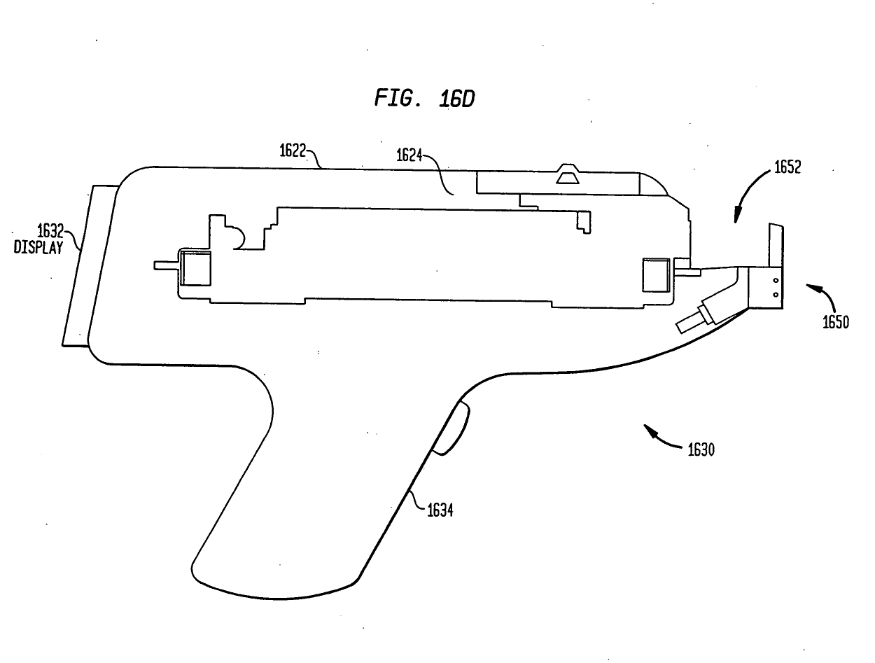

FIG. 16A schematically illustrates another embodiment of a handpiece device 1620 according to the teachings of the invention that includes a housing 1609 having a head for delivering energy generated by an energy source 1602 disposed in the housing to a target treatment area. More particularly, a beam formation system 1603, e.g., a lens, disposed in the housing directs the energy generated by the energy source 1602 onto the treatment area 1601. The energy source 1602 can be, for example, a radiation source, such as a laser, a lamp or an LED. Alternatively, the energy source can be a particle source, such as a dermal abrasion particle source. An illumination source 1606, for example, an LED, illuminates the treatment area, or a selected portion thereof. At least a portion of light reflected from the treatment area, for example, in response to illumination by the illumination source 1606, is imaged by a focusing or optical system 1604, for example, a lens, onto an image capture device 1605, e.g., a CCD camera, that generates an image of the treatment area. The image is transmitted to a display device 1608, mounted onto the housing, for viewing by a user.

The exemplary handpiece 1620 further includes a contact cooling window 1607 that thermally couples to a selected portion of the patient's skin so as to cool the patient's epidermis in the area of the skin exposed to treatment energy as the energy is deposited into a target treatment region. The cooling window 1607 is substantially transparent to both the treatment energy as well as the optical radiation generated by the energy source 1602.

FIG. 16B schematically illustrates that a memory unit 1610 can be incorporated in the handheld device 1620 for storing images obtained by the image capture device 1605. In addition, a communications interface 1611 allows the device to communicate, for example, via a wireless protocol, with an external computer. The communications interface 1611 allows for the transfer of images or data obtained by the image capture device 1605 to the external computer 1612, either in real time, or with a selected delay via downloading images stored by the memory unit 1610 onto the computer. Those having ordinary skill in the art will appreciate that other components, such as, processors, can also be included in the device to perform desired tasks.

A variety of designs can be employed for constructing a handheld dermatological device according to the teachings of the invention, such as the device schematically depicted above in FIGS. 4A and 4B. By way of example, FIG. 16C schematically illustrates a handheld dermatological device 1620 according to another embodiment of the invention that includes a housing 1622 having an enclosure 1624 in which various components of the device, for example, a radiation source, such as solid state laser together with optics for delivering radiation to a treatment area, and a CCD camera 1626 for imaging the treatment area, are disposed. The enclosure includes a treatment head 1628, such as a contact tip, at a proximal end thereof for delivering treatment energy to a selected target area, for example, via a window 1630 that can also function as a cooling element. Further, a display 1632 is mounted to the enclosure at a distal end thereof that allows a user to view the images captured by the CCD camera 1626. The housing 1622 further includes a handle 1634 that allows a user, e.g. a medical professional, a home user, or a beautician, to hold and manually manipulate the device for delivering treatment energy to a target area.

With reference to FIG. 16D, a handheld dermatological device 1630 according to another embodiment of the invention includes a housing 1622 formed of an enclosure 1624 and a handle 1634. The enclosure 1624 includes an optically transparent element 1650 mounted to a head portion thereof through which treatment radiation can be delivered to a target area. The enclosure further includes an opening 1652 that allows a user to directly view, via the transparent element 1650, a target area, albeit at a slanted viewing angle. Natural light from the sun, a cabinet lamp, or a head lamp/LED projector can be used for illumination of treatment area through opening 1652 to provide natural color of the skin. In addition, a display 1632 is mounted to the housing to allow the user to view an image of the treatment area obtained by an image capture device (not shown) incorporated in the housing.

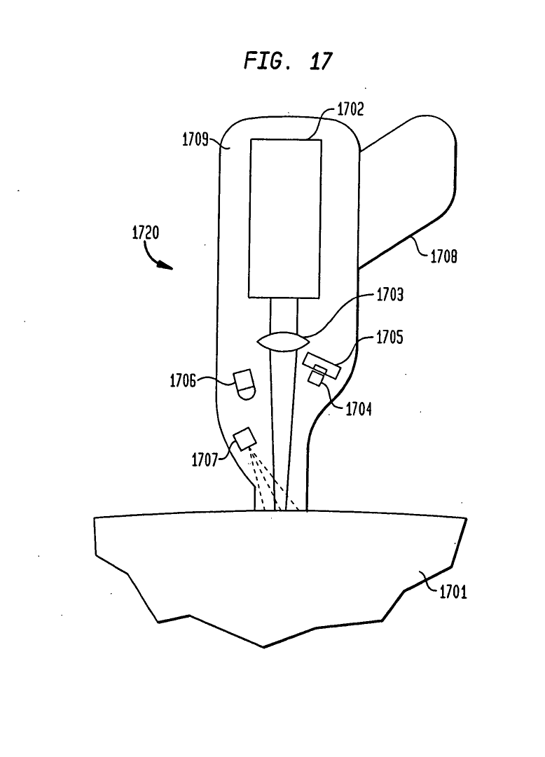

FIG. 17 schematically illustrates another embodiment of a handheld dermatological device 1720 according to the teachings of the invention that includes an energy source 1702 for generating treatment energy, e.g., electromagnetic, acoustic radiation or dermal abrasion particles, and a beam forming system 1703 for focusing the energy onto a selected target area inside a housing 1709. One or more illumination sources 1706 can illuminate the target area to allow an image capture device 1705 to obtain images of this area for presentation to a user via a display 1708 mounted to the housing. This embodiment further includes a non-contact cooling system 1707 for cooling the target area, e.g., during application of the treatment energy. The non-contact cooling system can be, among other choices, a spray unit that sprays a suitable coolant onto the treatment area, or it can be a system for generating an air flow over the treatment area. In this case, the imaging system can also be used to control cooling of the skin by a spray, for example, by monitoring for ice formation or “lake effects,” to prevent skin from over or under cooling.

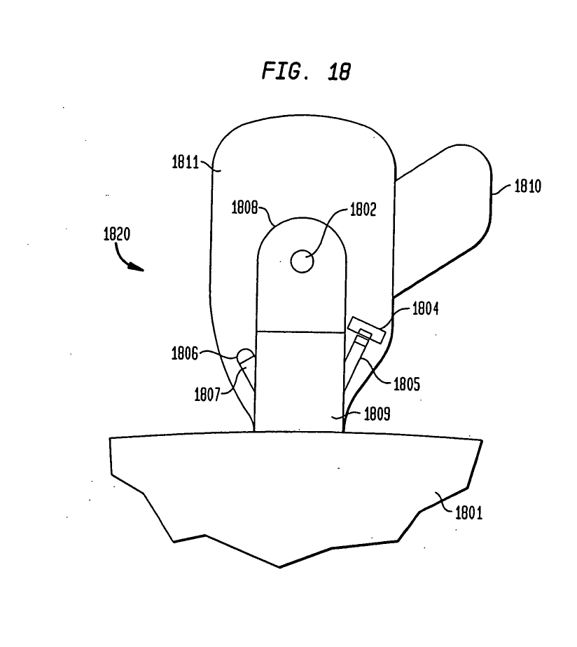

FIG. 18 schematically illustrates another handheld dermatological device 1820 according to another embodiment of the invention that includes a housing 1811 for enclosing a source 1802 (e.g., an arc, a halogen, a metal halide lamp) or solid state lighting sources (LED) 1802 for generating radiation, e.g., broadband radiation, and a reflector 1808 that directs at least a portion of the generated radiation to a the target treatment area or to a waveguide or an optically transparent window 1809 for delivery to the target treatment area. The exemplary device 1820 further includes a illumination source 1806, such as an LED, a laser, or a microlamp, that illuminates the target treatment area, or a portion thereof, via an optical coupling system 1807, e.g., a lens or prism. An optical coupling system 1805, e.g., a lens, focuses light reflected from the treatment area onto an image capture device 1804, e.g., a CCD camera, mounted to the device's housing for generating an image of the treatment area. The image is transmitted to a display 1810, mounted to the housing 1811, for viewing by a user. In some embodiments, one or more filters can be optically coupled to the lamp in order to select one or more wavelength bands from a broad spectrum generated by the lamp for causing desired therapeutic and/or cosmetic effects. The light from source 1802 can be used for illumination of the treatment area. Several sources like lamps 1802 can be mounted in the same reflecting chamber. Illumination sources 1806 can be mounted around skin treatment region or waveguide 1809 to provide illumination of a treatment target by banana photons. The light from illumination sources 1806 can be directly coupled to the skin. Further, a shield between illumination source 1806 and the observation skin region can be used. The depth of the illuminated area and the visualization depth into the skin can be optimized by control of incident angle of the illumination light on the 1809 contact surface, the observation angle of the optical coupling system 1805, refractive indices of the waveguide 1809, optical systems 1805 and 1807 and of a lotion, if utilized, between waveguide 1809 and the skin in a manner similar to that described above in connection with FIGS. 13A and 13B.

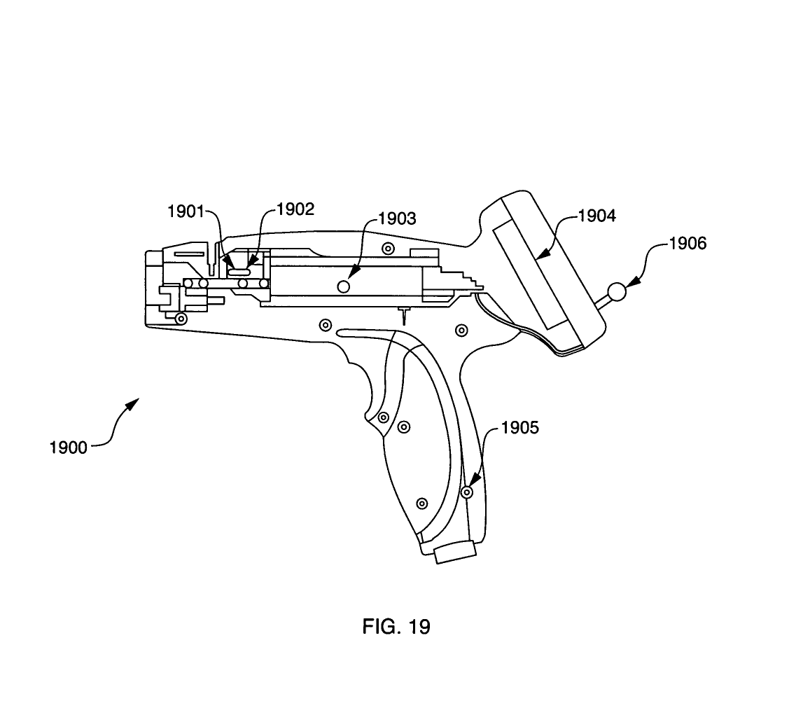

FIGS. 19-22C schematically illustrate another embodiment of a handheld dermatological device 1900 according to the invention. This device 1900 can include many of the features of the embodiment of FIGS. 4A-4C. The device 1900 can also be used for treatment of the same conditions or similar conditions as for previous embodiments, such as, e.g., hair removal, acne treatment, skin rejuvenation, blood vessel treatment, or tattoo removal.

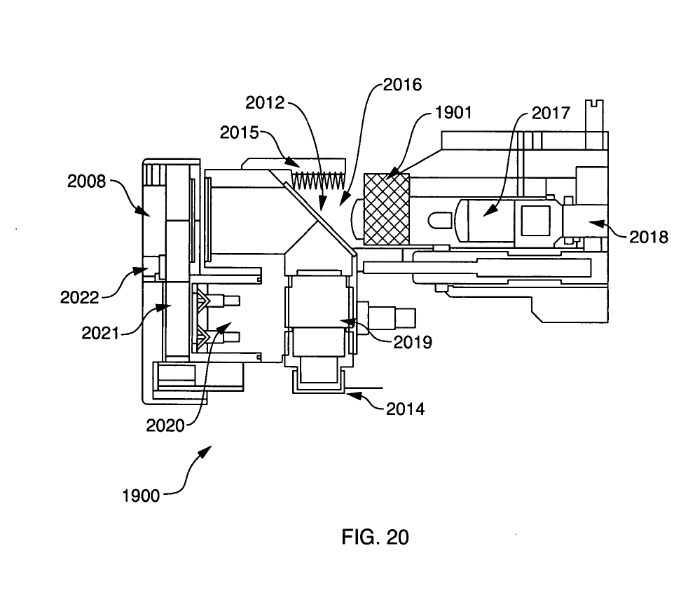

FIG. 19 is a schematic cross-sectional view of the handheld dermatological device 1900 according to this embodiment of the invention. The handheld device 1900 of FIG. 19 includes a treatment radiation source 1903, zoom optics 1902, a display 1904, and a handle 1905, all of which can be similar to those set forth above in connection with FIGS. 4A-4C. For example, the treatment radiation source 1903, disposed in the body portion of the handheld device 1900, generates radiation having one or more wavelengths suitable for treating a dermatological condition in the target skin region. The zoom optics 1902 or zoom assembly can include three lenses, one of which can move axially (i.e., along a direction of propagation of the treatment radiation beam) within a slider element relative to the other lenses so as to change the cross-sectional diameter of the treatment radiation beam. The display 1904 can be, e.g., a LCD display in which an image of the target region can be presented. The user of the handheld device 1900 can manually manipulate it by using the handle 1905 or hand piece to place its head portion in proximity of a subject's skin surface. As shown in FIG. 19, the device 1900 of this embodiment also includes a deflector or deflective optic 1901 and a control device 1906 for the deflective optic 1901, which are described in detail below in connection with FIG. 20.

FIG. 20 is an enlarged cross-sectional view of the left-hand side of the embodiment of FIG. 19. FIG. 20 illustrates the treatment radiation source 1903 and zoom optics 1902 of FIG. 19 in greater detail, as designated by numerals 2018 and 2017, respectively. FIG. 20 also illustrates a beam dump 2015 and a snap-in focusing lens 2016, which are in the optical path of the laser beam. The beam dump 2015 and lens 2016 can be of any variety known to those skilled in the art.

As shown in FIG. 20, the device 1900 includes a treatment window 2008 at the tip of the head portion. The treatment window 2008 defines a target treatment area of the tissue (such as the subject's skin) and is the portion of the device 1900 that contacts the tissue during use. The device 1900 can also include an illumination ring 2022, which can be utilized to illuminate the target region of the subject's skin located at a depth below the skin surface, as discussed in more detail above. The illumination ring 2022, for instance, can illuminate the area of the subject's skin adjacent the treatment window 2008. The illustrative device 1900 further includes an image capture device 2014, e.g., a CCD camera, for generating an image of the target region of the subject's skin. More particularly, tissue scattered radiation emanating from the target skin region can be directed by the beam splitter 2012 to a camera focusing lens 2019, which in turn focuses the image onto the image capture device 2014. The captured image can be displaced to the user on the display 1904 (FIG. 19).

The device 1900 can also include a cooling device or cooling tip 2021 that provides for the extraction of heat from a portion of the skin surface that can be in thermal contact therewith before, during, or after application of treatment radiation. FIG. 20 depicts heat exchangers 2020, such as connections to a cooling water supply, which can be used to cool the cooling tip 2021.

FIGS. 19 and 20 illustrate a deflective optic 1901 incorporated within the handheld device 1900. In operation, the deflective optic 1901 can steer the laser beam to change its position within the target region of the subject's skin. In one embodiment, the deflective optic 1901 can be a mechanical device that steers the laser beam. In this embodiment, for instance, the position of the deflective optic 1901 can be controlled in order to change the target position of the laser beam. In alternative embodiments, the deflective optic 1901 can be an electro-optic or acousto-optic deflector or any other suitable laser positioning mechanism.

The deflective optic 1901 can be, for example, an optical device that has its angular location varied by a mechanical motor. For instance, a mechanical motor can position the optic 1901 such that radiation traveling through the optic 1901 is steered to a new location within the target treatment area. Therefore, the deflective optic 1901 can be used to selectively steer radiation to a desired location within a target treatment area.

Referring to FIG. 19, the user can control the position of the laser beam on the subject's skin by controlling the deflective optic 1901 with the control device 1906. FIG. 19, for instance, depicts a joystick that is linked to the deflective optic 1901 and can be used to control the deflective optic 1901. In alternative embodiments, the control device 1906 can be a roller, a touch pad, a keypad, or any other type of device to control the deflective optic 1901.

FIG. 20 depicts the deflective optic 1901 disposed between the output of the zoom optics 2017 and the beam splitter 2012. In alternative embodiments, the deflective optic 1901 can be located in a different position within the handheld device 1900, such as, for example, between the beam splitter 2012 and the cooling tip 2021 or treatment window 2008. In another alternative embodiment, the deflective optic 1901 can be incorporated within the zoom optics 2017.

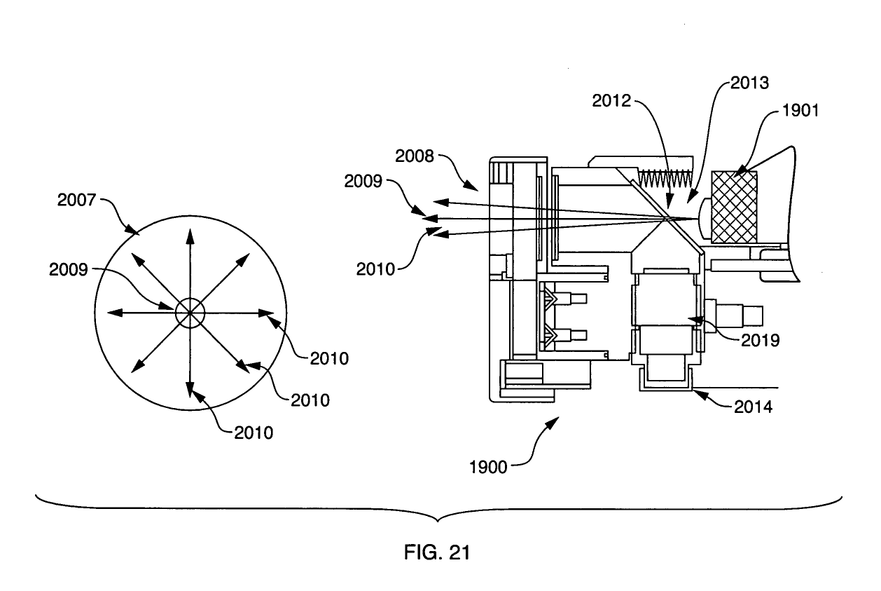

FIG. 21 depicts the handheld device 1900 of FIGS. 19 and 20 in use. In operation, the treatment window 2008 of the handheld device is brought into contact with a subject's skin. The treatment window 2008 of the device 1900 defines a target treatment area 2007 on the subject's skin, which is illustrated on the left-hand side of FIG. 21. This target treatment area 2007 can, in one embodiment, be displayed on the display device 1904 (FIG. 19) or any of the other display devices described above. When the deflective optic 1901 of the device 1900 is in a neutral or centered position, a centered laser beam 2009 results within the target treatment area 2007. This centered laser beam 2009 defines the area of the subject's skin (a treatment location) that the laser irradiates when applied. If the user wishes to change the position of the laser beam within the target treatment area 2007, the user can use the control device 1906 (FIG. 19) to adjust the deflective optic 1901, which deflects the laser beam. FIG. 21, for instance, depicts deflected beams 2010, each representing different control of the deflective optic 1901. A deflected beam 2010 can be deflected from the position of the centered laser beam 2009 in any circumferential and/or radial position within the target treatment area 2007, as illustrated on the left-hand side of FIG. 21. For instance, the deflective optic 1901 may deflect the beam upward so that the laser will irradiate a target position in the upper portion of the target treatment area 2007 of FIG. 21. Similarly, the beam may also be deflected downward so that the laser will irradiate a target position in the lower portion of the target treatment area 2007.

In another embodiment of the invention, the deflective optic 1901 can change the depth of focus within the target treatment area 2007. This feature can be used in conjunction with changing the location of the beam, or, alternatively, as a separate feature of the deflective optic 1901.

As described in connection with previous embodiments of the invention, in some embodiments, the display 1904 of the device 1900 can present not only an image of the target skin region or target treatment area 2007, but it can also include a graphical element, e.g., a circle or a cross-hair, that schematically depicts the cross-section of the treatment beam relative to the target region. For example, with reference to FIGS. 19 and 21, the display 1904 of the device 1900 could display a circle representing where the beam 2009 will strike the subject's skin. Such a display can appear, for example, as the circle designated by numeral 2009 for the centered beam on the left-hand side of FIG. 21. Such alignment features can significantly increase efficacy and safety of the treatment. For example, in the absence of such features, it is difficult to position small treatment beams (e.g., spot size less than 3 mm) on small treatment targets, such as blood vessels.

In one embodiment of the invention, when the user uses the control device 1906 to control the deflective optic 1901 and steer the beam, the device 1900 can update the position of the graphical element indicating where the beam will strike the subject's skin. Thus, with reference to FIG. 21, if the user controls the deflective optic 1901 so that the beam will strike toward the top of the target treatment area 2007, the graphical element on the display 1904 can be updated to depict the proper location within the target treatment area 2007 where the laser will strike the subject's skin. In some embodiments, the device 1900 can include circuitry to determine the position where the beam will strike the subject's skin based upon the control of the deflective optic 1901. Thus, the user of the device 1900 can control the deflective optic 1901, view the graphical element that indicates where the beam will strike the subject's skin, and then fire the radiation source 1903 to treat the subject's skin. Alternatively, after viewing the graphical element that indicates where the beam will strike the subject's skin, the user can reposition the deflective optic 1901 prior to firing the radiation source 1903. The control device 1906 can, in alternative embodiments, be located anywhere within the handheld device 1900, including, for example, in the handle 1905. In another alternative embodiment, the control device 1906 can be located remotely from the handheld device 1900, including, for example, on a base unit or on a foot pad or in a separate handheld mechanism.

In operation, the embodiment of the invention described above in connection with FIGS. 19-21 allows the user of the handheld device 1900 to control the position of the laser beam within a target treatment area 2007 on the subject's skin. Thus, during use, the user does not need to lift the device off the subject's skin, reapply the device to a new location on the subject's skin, and then fire the laser to treat a different spot on the skin within the target treatment area 2007. Instead, the user can treat any area within the target treatment area 2007 by controlling the deflective optic 1901, and hence the laser beam, without having to reposition the handheld device 1900 on the subject's skin.

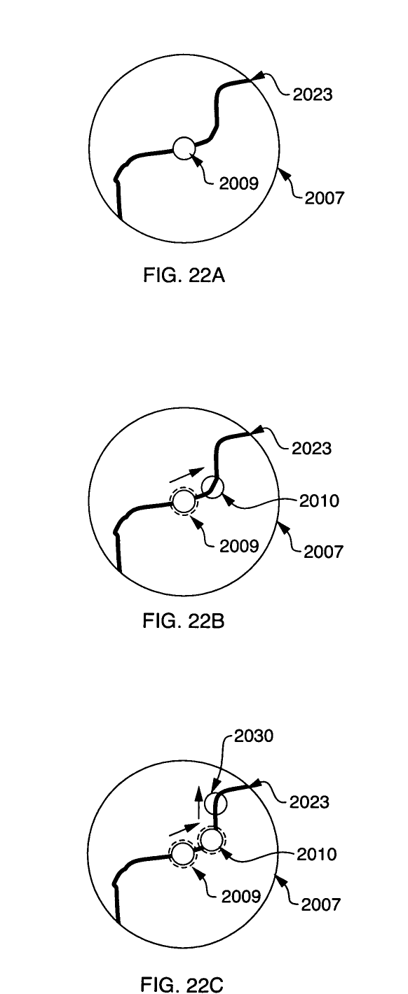

FIGS. 22A-22C illustrate the device 1900 of FIGS. 19-21 in operation. FIG. 22A depicts a target treatment area 2007 containing a blood vessel 2023. Although FIG. 22A depicts a blood vessel 2023, the device 1900 can be used for the treatment of numerous other medical and cosmetic conditions, as set forth above. The circle indicated with numeral 2009 indicates a centered laser beam within the target treatment area 2007. The user could fire the laser to treat the blood vessel 2023 at the location and/or reposition the beam to another location. In FIG. 22B, the position of the laser beam has been adjusted through manipulation of the deflective optic 1901. The deflected beam will strike the subject's skin in the area designated by numeral 2010. Again, the user can fire the laser at this location and/or reposition the beam to another location. Thus, in this example, the user of the device 1900 adjusted the position of the laser beam from FIG. 22A to FIG. 22B in order to treat two different areas of the blood vessel 2023 within the target treatment area 2007. In this embodiment, the user accomplished this task without adjusting the position of the device 1900 itself on the subject's skin. Instead, the user made the adjustment by controlling the deflective optic 1901 to reposition the location of the laser beam. FIG. 22C illustrates another adjustment of the deflective optic 1901 in order to treat another area of the blood vessel 2023 within the target treatment area 2007. In the embodiment of FIG. 22C, the deflected beam will strike the subject's skin in the area designated by numeral 2030. Thus, through adjustment of the deflective optic 1901, the user can reposition the centered beam location within the target treatment area 2007 from location 2009 to 2010, and further to location 2030, as shown by the arrows in FIGS. 22B and 22C.

As set forth in connection with previous embodiments, in some embodiments, the handheld device 1900 can track the position of a target region, e.g., a treatment site, which can be identified by a marker in an image, from one image to the next. For example, with reference to FIG. 22A-22C, a marker can be provided to identify treated areas of the skin. FIGS. 22B and 22C, for instance, show broken circles for the areas that have already been treated.

In another embodiment of the invention, the image capture device (e.g. a camera) 2014 (FIG. 20) of the device 1900 can capture an image within the target treatment area 2007 of a condition for treatment, such as the blood vessel 2023 of FIGS. 22A-22C. The device 1900 can then use object detection software in a computer of the device 1900 to determine or trace out the shape of the condition for treatment. Based on this information, the computer can calculate how the deflective optic 1901 should be adjusted to move the laser beam along the condition to treat the condition. For instance, the computer can calculate a first treatment location, a second treatment location, and subsequent treatment locations so that the entire condition within the target treatment area 2007 can be treated through computer control without requiring the user to reposition the beam prior to application of radiation.

In the example of FIGS. 22A-22C, the device can use the image capture device (e.g. a camera) 2014 to determine the shape of the blood vessel 2023 within the target treatment area 2007, and it can then calculate how the deflective optic 1901 should be moved to treat different parts of the blood vessel, such as, for example, shown in FIGS. 22A, 22B, and 22C.

Thus, in this embodiment, a computer incorporated within the device 1900 can be used to automate the treatment process. In other embodiments, the computer can also determine when to fire the radiation source 1903 after positioning of the beam through adjustment of the deflective optic 1901. Further, in other embodiments, the computer can determine how long to fire the radiation source 1903 for treatment of the condition. Thus, through the use of the image capture device (e.g. a camera) 2014 to capture an image of the condition, the deflective optic 1901, and a computer to control the position of the deflective optic 1901 and firing of the radiation source 2003, treatment of a condition within the treatment window 2007 can be automated.

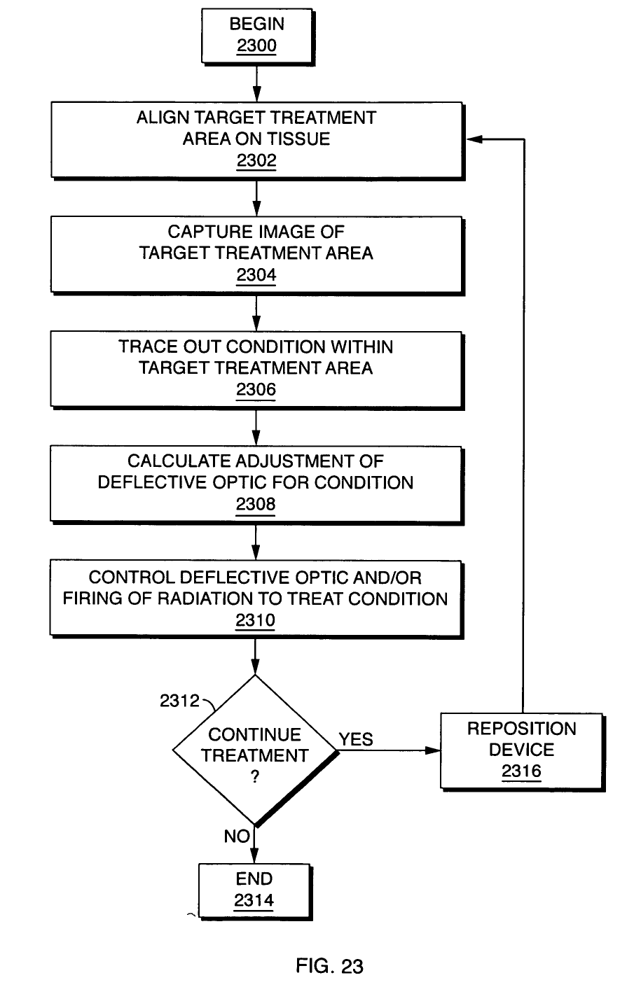

FIG. 23 is a flow chart of treatment of tissue during use of the dermatological device 1900 of FIG. 19 according to one embodiment of the invention. At block 2300, an operator begins a treatment process. The operator positions the device 1900 on tissue to define a target treatment area on the tissue, as shown at block 2302 of FIG. 23. The image capture device 2014 then captures an image of the target treatment area, including a condition on the tissue for treatment, as shown at block 2304. Next, at block 2306, object detection software in a computer of the device 1900 traces out or calculates the shape of the condition for treatment within the target treatment area. That is, the software calculates positional information for the condition. The computer then calculates how to adjust the deflective optic 1901 in order to treat the condition as traced out, which is shown at block 2308. This can include determining how to adjust the deflective optic 1901 to steer the radiation in order to treat multiple treatment locations of the condition within the target treatment area. In addition, this step can also involve determining when to fire the radiation source 1903 after adjustment of the deflective optic 1901. Next, at block 2310, the deflective optic 1901 and firing of the radiation source 1903 are controlled by the computer of the device 1900 in order to treat the treatment location of the condition. For instance, the deflective optic 1901 can be steered to a first treatment location, fired, then steered to a second treatment location and fired, and so on, in order to treat the location within the target treatment area.

After the condition within the target treatment area has been treated, the operator can decide whether or not to continue treatment of the condition in areas outside the target treatment area. This is shown at block 2312 of FIG. 23. If the operator does not wish to continue treatment, the procedure is done (block 2314). If, on the other hand, the operator wishes to continue treatment of the condition, the operator can reposition the device 1900 to form a new target treatment area on the tissue. This is shown at block 2316. The procedure can then recommence at block 2302 in order to treat the condition within the new target treatment area on the tissue.

In FIG. 23, steps 2300, 2302, 2312, 2314, and 2316 are steps carried out by the operator of the device 1900. On the other hand, after proper programming and selection of options of the device 1900, steps 2302, 2304, 2306, 2308, and 2310 can be automatically carried out by the device 1900 upon placement of the device 1900 on tissue. The computer of the device 1900 can be programmed for a variety of treatment conditions (i.e., blood vessel treatment, acne treatment, etc . . . ) so that the proper radiation wavelength and firing time of the radiation source 1903 can be calculated. In addition, the computer can be programmed to recognize certain properties of conditions in order to properly trace out the condition. Thus, the operator of the device 1900 need only select proper target treatment areas on the tissue, and the rest of the procedure can be automatically performed by the device 1900.

While several embodiments of the invention have been described and illustrated herein, those of ordinary skill in the art will readily envision a variety of other means and structures for performing the functions and/or obtaining the results and/or advantages described herein, and each of such variations or modifications is deemed to be within the scope of the present invention. More generally, those skilled in the art would readily appreciate that all parameters, dimensions, materials, and configurations described herein are meant to be exemplary and that actual parameters, dimensions, materials, and configurations will depend upon specific applications for which the teachings of the present invention are used. Those skilled in the art will recognize, or be able to ascertain using no more than routine experimentation, many equivalents to the specific embodiments of the invention described herein. The present invention is directed to each individual feature, system, material and/or method described herein. In addition, any combination of two or more such features, systems, materials and/or methods, if such features, systems, materials and/or methods are not mutually inconsistent, is included within the scope of the present invention.

1. An apparatus for performing a treatment on tissue, comprising:
a housing having a portion that defines a target treatment area on the tissue when located in proximity to the tissue;
a radiation source for generating radiation; and
a deflector to selectively steer radiation from the radiation source to a treatment location within the target treatment area.

2. The apparatus of claim 1, wherein the deflector comprises an optical device.

3. The apparatus of claim 1, wherein the deflector comprises a mechanical motor and an optic device, wherein the mechanical motor can adjust an angular position of the optic device to steer radiation.

4. The apparatus of claim 1, further comprising an illumination source for illuminating the target treatment area on the tissue.

5. The apparatus of claim 1, further comprising a control device for controlling the deflector.

6. The apparatus of claim 1, further comprising a detector to detect radiation emanating from the tissue.

7. The apparatus of claim 6, further comprising a first polarizer coupled to the radiation source and a second polarizer coupled to the detector.

8. The apparatus of claim 6, wherein the detector is an image capture device for generating an image of the illuminated tissue in the target treatment area.

9. The apparatus of claim 8, further comprising a display device to display the image.

10. The apparatus of claim 9, further comprising an image processor in communication with the image capture device and the display device.

11. A handheld dermatological device for performing a treatment on tissue, comprising:
a housing having a head portion that defines a target treatment area on the tissue when located in proximity to the tissue;
a radiation source for generating radiation;
an image capture device for capturing an image of the target treatment area;
a display, operably coupled to the housing, to display the image;
a deflector to steer radiation from the radiation source to a treatment location within the target treatment area; and
a user control device to control the deflector.

12. A method of operating a handheld dermatological device, comprising:
capturing an image of a condition on tissue through an image capture device of the handheld dermatological device, wherein the image of the condition is of at least a portion of the condition in a target treatment area defined by a head portion of the handheld dermatological device when located in proximity to the tissue; and
steering radiation to selectively treat the condition within the target treatment area.

13. The method of claim 12, wherein the act of steering comprises steering radiation through adjustment of a deflector.

14. The method of claim 13, wherein the deflector comprises a mechanical motor and an optic device, wherein the mechanical motor can adjust an angular position of the optic device to steer radiation.

15. The method of claim 12, further comprising controlling the steering of the radiation through a user control device.

16. The method of claim 12, further comprising displaying the image to an operator of the handheld dermatological device.

17. The method of claim 16, wherein displaying the image includes displaying a graphic to represent where the radiation will strike the tissue.

18. The method of claim 12, further comprising displaying an image of the condition to an operator of the handheld dermatological device.

19. The method of claim 12, further comprising illuminating the target treatment area to aid in capturing the image.

20. The method of claim 12, further comprising calculating positional information of the condition within the target treatment area.
